(19) United States
(12) Patent Application Publication
(10) Pub. No.: US 2025/0179568 A1
(43) Pub. Date: Jun. 5, 2025

(54) Target Enrichment

(71) Applicant: New England Biolabs, Inc., Ipswich, MA (US)

(72) Inventors: Cynthia Hendrickson, Wenham, MA (US); Kruti Patel, Ipswich, MA (US)

(73) Assignee: New England Biolabs, Inc., Ipswich, MA (US)

(21) Appl. No.: 18/843,841

(22) PCT Filed: Mar. 8, 2023
(86) PCT No.: PCT/US2023/063895

§ 371 Date: Sep. 4, 2024

Related U.S. Application Data
(60) Provisional application No. 63/318,194, filed on Mar. 9, 2022.

(51) Int. Cl. C12Q 1/6855; C12N 15/10; C12Q 1/6806; C12Q 1/6834

(52) U.S. Cl. CPC C12Q 1/6855; C12N 15/1065; C12Q 1/6806; C12Q 1/6834

(57) ABSTRACT

The present disclosure relates, according to some embodiments, to methods and compositions for preparing polynucleotide libraries enriched for a target sequence from source materials (e.g., samples comprising or constituting biological fluids, tissues, and/or specimens). Methods and compositions may provide efficient enrichment of the targeted polynucleotide. Enrichment may include increasing the relative abundance of the target from the source materials (where it may be present in low abundance) to the produced libraries (where it may be present in higher abundance). In some embodiments, methods include attaching (e.g., ligating, joining or otherwise fusing) an adapter to fragments of interest, nick translation, amplification (e.g., linear amplification), and target sequence selection using, for example, affinity tagging.

### CROSS REFERENCE TO RELATED APPLICATION

This application claims the benefit of provisional application Ser. No. 63/318,194, filed on Mar. 9, 2022, which application is incorporated by reference herein in its entirety.

### SEQUENCE LISTING STATEMENT

This disclosure includes a Sequence Listing submitted electronically in .xml format under the file name “DG-004-PCT.xml” created on Mar. 1, 2023, and having a size of 15.6 KB. This Sequence Listing is incorporated herein in its entirety by this reference.

### BACKGROUND

Next-generation sequencing (NGS) has become a major tool in genomics research, providing a powerful way to study DNA samples. There is an intense effort to develop NGS-based methods for the analysis of genomic variation. As part of this effort, several methods have been developed that enrich for specific target sequences, e.g., sub-regions of a genome, to reduce costs and labor and to improve outcomes. Target enrichment may be helpful when only a portion of a genome needs to be enriched/analyzed, such as the ‘exome’ (i.e., all transcribed sequences) or smaller sets of genes or genomic regions that are implicated in a particular disease or pathway. For example, target enrichment may be used to select the DNA for a set of cancer genes prior to sequence analysis. Selectively recovering target sequences should, in theory, reduce cost and increase sequencing depth relative to whole-genome sequencing.

It may be challenging to perform target enrichment on low input samples, not only because there is a limited amount of DNA in the sample but also because the enrichment methods themselves are inefficient. Thus, there is still a need for new methods for target enrichment, particularly methods that can be used for low input samples such as cell-free DNA (cfDNA) and DNA that has been isolated from tissue sections.

### SUMMARY

The present disclosure provides methods for enriching polynucleotides comprising a sequence of interest (a “target sequence”) in a polynucleotide composition. In some embodiments, a polynucleotide composition may comprise genomic, cellular, organellar, cell free, or other polynucleotides (e.g., DNA and/or RNA) fragmented, for example, to form a population of fragments with at least some (optionally, all) having sizes within a desired range of fragment sizes. A population of fragments may include one or more fragments having a target sequence. A method, according to some embodiments, may comprise attaching (e.g., ligating, joining or otherwise fusing) a DNA adapter to 5′ ends of double-stranded polynucleotide fragments in a population of double-stranded polynucleotide fragments from a sample, wherein the DNA adapter comprises a bottom strand and a top strand and fusion products are formed. An adapter top strand may comprise, from 5′ to 3′, a sequence of at least 8 nucleotides that is complementary to a linear amplification annealing site, optionally, a sample tag, optionally, a unique molecule identifier (UMI), and a sequence that is complementary to the adapter bottom strand. An adapter top strand may be longer than an adapter bottom strand, for example, where the optional sample tag and/or the optional UMI are included in the top strand. An adapter bottom strand may comprise a non-extendible 3′ end (e.g., an end comprising an inverted deoxythymidine). An adapter bottom strand may not include (may exclude) a 5′ phosphate, a sample tag, a UMI and/or a linear amplification annealing site. In some embodiments, fusion products may comprise 3′ ends of the adapter top strands fused to 5′ ends of the polynucleotide fragments and nicks comprising unfused 5′ ends of the adapter bottom strands and 3′ ends of the polynucleotide fragments.

In some embodiments, a method may comprise attaching (e.g., ligating, joining or otherwise fusing) a DNA adapter to 5′ ends of double-stranded polynucleotide fragments in a population of double-stranded polynucleotide fragments from a sample, wherein the DNA adapter comprises a top strand and optionally a bottom strand and fusion products are formed. A single-stranded adapter may be attached to double-stranded fragments using, for example, a single-strand DNA ligase. An adapter top strand may comprise, from 5′ to 3′, a sequence of at least 8 nucleotides that is complementary to a linear amplification annealing site, optionally, a sample tag, optionally, a unique molecule identifier (UMI), and a sequence that is optionally complementary to the adapter bottom strand. Including an optional bottom strand may be desirable where a top strand comprises a UMI (e.g., to reduce or prevent UMI hopping). In some embodiments, an adapter top strand may lack a UMI and a DNA adapter may lack a bottom strand. An adapter top strand may be longer than an adapter bottom strand, for example, where the optional sample tag and/or the optional UMI are included in the top strand. An adapter bottom strand may comprise a non-extendible 3′ end (e.g., an end comprising an inverted deoxythymidine). An adapter bottom strand may not include (may exclude) a 5′ phosphate, a sample tag, a UMI and/or a linear amplification annealing site. In some embodiments, fusion products may comprise 3′ ends of the adapter top strands fused to 5′ ends of the polynucleotide fragments and nicks comprising unfused 5′ ends of the adapter bottom strands and 3′ ends of the polynucleotide fragments.

A method may further comprise, according to some embodiments, contacting fusion products with a nick-translating polymerase for templated addition of nucleotides to the 3′ ends of the polynucleotide fragments to form nick translation products comprising 3′ extensions of the polynucleotide fragment, the extensions complementary to the adapter top strands. A method may additionally comprise contacting the nick translation products with a primer having a sequence complementary to the linear amplification annealing site and a polymerase to produce amplified (e.g., linearly amplified) nick translation products. Amplified (e.g., linearly amplified) nick translation products may comprise, for example, in a 5′ to 3′ direction, a sequence complimentary to the linear amplification annealing site, optionally, a sequence of the sample tag, optionally, a sequence of the UMI, a sequence of a portion of the adapter top strand, a sequence of one of the polynucleotide fragments, and optionally, a sequence complementary to a portion of the adapter top strand (e.g., 1-25 nucleotides).

According to some embodiments, a method may further comprise, prior to or concurrent with amplification (e.g., linear amplification), contacting nick translation products with a glycosylase and an endonuclease (e.g., serially, together as separate enzymes, or as a fusion protein), wherein the adapter top strand template further comprises one or more modified nucleotides, wherein the one or more modified nucleotides are optionally deoxyuridine, the glycosylase is optionally uracil-DNA glycosylase (UDG), and the endonuclease is optionally endonuclease VIII. Nick translation may be expected to displace the adapter bottom strand and such displaced adapter bottom strand may be expected to be lost, for example, through subsequent steps without any overt action required. In some embodiments, however, a method may comprise removing the adapter bottom strand of the adapter template (after nick translation) by cleaving the adapter bottom strand with a reagent comprising a glycosylase and an endonuclease, wherein the adapter bottom strand comprises one or more modified nucleotides, wherein the one or more modified nucleotides are optionally deoxyuridine, the glycosylase is optionally uracil-DNA glycosylase (UDG), and the endonuclease is optionally endonuclease VIII.

A method may further comprise, according to some embodiments, contacting linearly amplified nick translation products with a target-specific oligonucleotide attached to an affinity domain (e.g., biotin) to form target complexes comprising polynucleotide fragments having the target sequence, the target-specific oligonucleotide, and the affinity domain, wherein the target sequence is hybridized to the target-specific oligonucleotide. In some embodiments, a method may include binding target complexes to a solid support comprising an affinity capture domain (e.g., streptavidin) corresponding to the affinity domain.

In some embodiments, a method may include removing any overhanging polynucleotide sequence at the 3′ end of the oligonucleotide hybridized complementary strand using a 3′-5′ single strand exonuclease or a plurality of 3′-5′ exonucleases for forming a flush end duplex of the 3′ end of the complement and the 5′ end of the oligonucleotide; attaching (e.g., ligating, joining or otherwise fusing) the flush end duplex to a second adapter comprising a second sample tag to form second fusion products comprising, in a 5′ to 3′ direction, the first adapter top strand, the oligonucleotide fragment, and the second adapter; and/or amplifying the second fusion products for sequencing of one or both of the strands.

It may be desirable, in some embodiments, to combine polynucleotide compositions for multiplex analysis. For example, polynucleotide compositions (e.g., comprising genomic, cellular, organellar, cell free, or other polynucleotides from the same or different samples, specimens, cells, fluids, tissues, organisms, or other materials) may be combined prior to fragmentation or at any step of methods disclosed here, for example, after adapter fusion or after linear amplification. Accordingly, a method may comprise, in some embodiments, combining adapter fusion products arising from a plurality of separate polynucleotide compositions (e.g., comprising genomic, cellular, organellar, cell free, or other polynucleotides from the same or different samples, specimens, cells, fluids, tissues, organisms, or other materials). For example, a method may comprise pooling linear amplification products (e.g., from a first adapter fusion, nick translation, and linear amplification) with further linear amplification products (e.g., from a second adapter fusion, nick translation, and linear amplification), the further linear amplification products each comprising, in a 5′ to 3′ direction, a sequence complimentary to a linear amplification annealing site, the sequence of a further sample tag, optionally, a sequence of the UMI, a sequence of a portion of a further adapter top strand, and a sequence of a polynucleotide fragment of a further population of fragments.

In some embodiments, a method may comprise repeating the adapter fusion step with a second DNA adapter and double-stranded polynucleotide fragments of a second fragment population to form further fusion products, wherein the first adapter top strand comprises a sample tag, and wherein the second adapter comprises a bottom strand and a top strand (e.g., a longer top strand). A second adapter top strand may comprise, from 5′ to 3′, a sequence of at least 8 nucleotides that is complementary to the linear amplification annealing site, a sample tag that differs from the first adapter sample tag, optionally, a unique molecule identifier (UMI); and a sequence that is complementary to the second adapter bottom strand. A second adapter bottom strand may comprise a non-extendible 3′ end and optionally may contain no 5′ phosphate, no sample tag, no UMI, and no linear amplification annealing site.

In some embodiments, polynucleotide fragments of the second population may comprise a target sequence. Further fusion products may comprise 3′ ends of the second adapter top strands fused to 5′ ends of the polynucleotide fragments of the second population and nicks comprising unfused 5′ ends of the second adapter bottom strands and 3′ ends of the polynucleotide fragments of the second population. A method may comprise pooling the fusion products and the further fusion products to form pooled fusion products.

In some embodiments, the step of contacting the fusion products with the nick-translating polymerase may further comprise contacting the pooled fusion products with the nick-translating polymerase for templated addition of nucleotides to the 3′ ends of the polynucleotide fragments to form pooled nick translation products comprising 3′ extensions of the polynucleotide fragments, the extensions complementary to the respective adapter top strands. The step of contacting the nick translation products with the primer having a sequence complementary to the linear amplification annealing site may further comprise, in some embodiments, contacting the pooled nick translation products with the primer and the polymerase to produce linearly amplified pooled nick translation products, wherein the linearly amplified pooled nick translation products may comprise, in a 5′ to 3′ direction, a sequence complimentary to the linear amplification annealing site, a sequence of one of the respective sample tags, optionally, a sequence of the UMI, a sequence of a portion of the respective adapter top strands, a sequence of one of the polynucleotide fragments, and optionally, a sequence complementary to a portion of the respective adapter bottom strands.

According to some embodiments, a method for enriching a target sequence may comprise contacting a population of fragmented polynucleotides with a ligase and a first adapter, the first adapter comprising a bottom strand and a top strand (e.g., a longer top strand), wherein (i) the bottom strand comprises, in a 5′ to 3′ direction, a 5′-OH, at least 10 nucleotides complementary to the top strand, optionally, and an inverted dT; and (ii) the top strand comprises, in a 5′ to 3′ direction, a sequence complementary to a linear amplification annealing site, optionally a sample tag, a unique molecular identifier (UMI), and a sequence complementary to the bottom strand, wherein the top and bottom strands optionally and independently comprise one or more deoxyuridines (e.g., distributed along the length of the respective strand) and wherein the contacting produces double stranded fusion products comprising 3′ ends of adapter top strands fused to 5′ends of polynucleotide fragments and nicks comprising unfused 5′ ends of adapter bottom strands and 3′ ends of polynucleotide fragments. In some embodiments, a method may further include contacting the double stranded fusion products with a nick-translating polymerase and nucleotide triphosphates to form nick-translated duplex polynucleotides wherein the duplex comprises a top strand comprising, in a 5′ to 3′ direction, the adapter top strand, the plus strand of one of the polynucleotide fragments, and a sequence complementary to the adapter top strand and a bottom strand comprising, in a 5′ to 3′ direction, the adapter top strand, the minus strand of one of the polynucleotide fragments, and a sequence complementary to the adapter top strand; and optionally contacting the nick-translated duplex polynucleotides with a single nucleotide excision reagent (e.g., that removes deoxyuridines) to form polynucleotide duplexes comprising a plus strand and a minus strand, each comprising single nucleotide gaps in the adapter top strand of the nick-translated duplex polynucleotides, the gaps defining oligonucleotides of varying lengths. A method may further include contacting the nick-translated duplex polynucleotides with a linear amplification primer having a sequence that is complementary to the linear amplification annealing site to form annealed duplexes, in some embodiments. A method may further include, according to some embodiments, contacting the annealed duplexes with a polymerase to form primer extension products comprising, in a 5′ to 3′ direction, a sequence corresponding to at least a portion of the adapter top strand, and a sequence corresponding to at least a portion of the plus strand of one of the polynucleotide fragments. Amplification (e.g., linear amplification) may be achieved by repeating the annealing and polymerization steps at least once (e.g., 5-10 cycles, 5-20 cycles, 5-30 cycles, 10-50 cycles, or >50 cycles). In some embodiments, a method may further include contacting the primer extension products with a target isolation probe comprising an affinity domain and a target-specific oligonucleotide to form a target probe mixture comprising the primer extension products, target isolation probes, and if one or more target sequences are present in the population of fragmented polynucleotides, target complexes comprising primer extension products having the target sequence hybridized to the target-specific oligonucleotides of the target isolation probes. A method may also include, according to some embodiments, contacting the target probe mixture with a solid support comprising affinity capture domains corresponding to the affinity domains of the target isolation probes to form affinity complexes comprising affinity domains bound to affinity capture domains. A method for enriching a target sequence may comprise separating affinity complexes from unbound primer extension products and unbound target isolation probes to form an enriched target sequence composition, in some embodiments.

A method, in some embodiments, may comprise A-tailing the fragmented polynucleotides prior to fusion with the first adapter, for example, where the top strand of the adapter further comprises a 3′ T or dU. A method may include (e.g., for multiplex analysis) repeating all method steps, wherein the population of fragmented polynucleotides of each repeated first adapter fusion step is different from all prior first adapter fusion steps and wherein the sample tag of each first adapter fusion step is different from all prior first adapter fusion steps, and optionally pooling the affinity complexes arising from each repetition (or cycle). Methods may include one or more optional washing steps to remove (at least some portion of) fragments the lack a target sequence, for example, after formation of target complexes and/or after formation of affinity complexes.

### BRIEF DESCRIPTION OF THE FIGURES

FIGS. 1A-1B shows schematic representations of example methods of forming enriched libraries of target sequences from complex (e.g., genomic) DNA samples. FIG. 1A illustrates a simple flowchart for an example enrichment workflow. FIG. 1B illustrates an example workflow.

FIGS. 2A-2B shows schematic representations of example methods of forming enriched libraries of target sequences from complex (e.g., genomic) DNA samples. FIG. 2A illustrates a simple flowchart for an example enrichment workflow. FIG. 2B illustrates an example workflow.

FIGS. 3A-3C illustrates sequencing results from three low input samples, each enriched for distinct targets, one totaling 75 kb (FIG. 3A), one totaling 227 kb (FIG. 3B), and one totaling 1100 kb (FIG. 3C).

### DETAILED DESCRIPTION

The present disclosure relates, in some embodiments, to methods and compositions for preparing polynucleotide libraries enriched for a target sequence from source materials (e.g., samples comprising or constituting biological fluids, tissues, and/or specimens). Methods and compositions may provide efficient enrichment of the targeted polynucleotide. Enrichment may include increasing the relative abundance of the target from the source materials (where it may be present in low abundance) to the produced libraries (where it may be present in higher abundance). In some embodiments, methods include attaching (e.g., ligating, joining or otherwise fusing) an adapter to fragments of interest, nick translation, amplification (e.g., linear amplification), and target sequence selection using, for example, affinity tagging.

Targeted genomic analysis applications may include, for example, the analysis of as little as one target of one or a few bases to identify a clinically actionable mutation. Such applications may include, alternatively, analysis of as much as a million targets (or more) consisting of 50-100 megabases to sequence an entire exome or an entire genome of a pathogen enriched from a host sample to discover variants of interest. Single to a few targets are typically captured by PCR methods while very large numbers of targets are efficiently captured by traditional hybridization capture of PCR amplified whole genome libraries. Intermediate sized panels, for highly sensitive interrogation of hundreds to thousands of targets are poorly addressed by these methods. PCR has limited multiplexing capacity, thereby requiring many separate reactions to cover target sets of this size. Traditional hybridization capture of target library fragments has reduced specificity at smaller panel size, due to the nonspecific capture of some off-target fragments. In addition, bias can be introduced during the PCR amplification prior to enrichment and polymerase errors introduced during PCR are amplified.

The present disclosure provides, in some embodiments, methods of capturing the advantages of existing hybridization techniques while increasing specificity by performing the hybridization capture of the target fragments before they are fully converted into a library with the necessary 3′ and 5′ fixed sequence and/or inhibiting conversion of off target fragments into full-length library molecules. In addition, the present disclosure provides methods including amplification that is required with low input samples to increase sensitivity but utilizes linear amplification, rather than PCR, to avoid introducing significant amplification bias and/or to avoid amplifying polymerase-introduced errors.

### General Considerations

Aspects of the present disclosure can be further understood in light of the embodiments, section headings, figures, descriptions and examples, none of which should be construed as limiting the entire scope of the present disclosure in any way. Accordingly, the claims set forth below should be construed in view of the full breadth and spirit of the disclosure.

Each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible. Except where otherwise noted, all reagents referenced in the present disclosure may be obtained from New England Biolabs, Inc. (Ipswich, MA).

Unless otherwise defined, all technical and scientific terms used herein have the same meaning as commonly understood by one of ordinary skill in the art to which this disclosure belongs. Still, certain terms are defined herein with respect to embodiments of the disclosure and for the sake of clarity and ease of reference.

Numeric ranges are inclusive of the numbers defining the range. All numbers should be understood to encompass the midpoint of the integer above and below the integer i.e., the number 2 encompasses 1.5-2.5. The number 2.5 encompasses 2.45-2.55 etc. When sample numerical values are provided, each alone may represent an intermediate value in a range of values and together may represent the extremes of a range unless specified.

Sources of commonly understood terms and symbols may include: standard treatises and texts such as Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); Singleton, et al., Dictionary of Microbiology and Molecular biology, 2d ed., John Wiley and Sons, New York (1994), and Hale & Markham, the Harper Collins Dictionary of Biology, Harper Perennial, N.Y. (1991) and the like.

As used herein and in the appended claims, the singular forms “a” and “an” include plural referents unless the context clearly dictates otherwise. For example, the term “a protein” refers to one or more proteins, i.e., a single protein and multiple proteins. It is further noted that the claims can be drafted to exclude any optional element. As such, this statement is intended to serve as antecedent basis for use of such exclusive terminology as “solely,” “only” and the like in connection with the recitation of claim elements or use of a “negative” limitation.

In the context of the present disclosure, an “adapter” refers to a polynucleotide configured to be attached (e.g., ligated, joined or otherwise fused) to a desired polynucleotide (e.g., a fragmented polynucleotide). An adapter may be linear, having two ends. One end may be suitable for ligation to other polynucleotides. For example, an end may be blunt (e.g., for blunt end ligation), have a single nucleotide overhang ligation (e.g., a T or U overhang for ligation to A-tailed polynucleotides), or have a longer overhang (e.g., 2-20 nucleotides for ligation to polynucleotides having complementary overhangs). An adapter may comprise a non-extendible 3′ end. For example, each 3′ end of an adapter may have an inverted dT (e.g., TriLink Biotechnologies), joined to the penultimate nucleotide by a 3′-3′ linkage that is resistant to extension by DNA polymerases. The other end of the polynucleotide (e.g., distal to the end selected or adapted for ligation) may be the same or different. When different, it may comprise a nucleotide of any length, any sequence, with or without complementarity to the rest of the adapter. For example, the end may comprise a long overhang without complementarity to a strand that otherwise pairs with the adapter. An adapter may comprise a single strand capable of forming a loop (e.g., comprising a hairpin) with at least a first portion of the adapter strand hybridized to a second portion of the adapter strand.

Double-stranded adapters may comprise a top strand and a bottom strand, wherein each strand comprises a separate oligonucleotide strand. Double-stranded adapters (e.g., loop adapters) may comprise a top strand and a bottom strand, wherein a single polynucleotide strand folds back on itself to provide both a top strand and a bottom strand (see, e.g., U.S. Pat. No. 8,288,097 and US 2012/0244525A1).

In the context of the present disclosure, an “affinity domain” refers to a domain capable of binding a corresponding affinity capture domain with high affinity (e.g., at least 10⁻⁸M) and specificity. Example materials having such properties include biotin, DBT, desthiobiotin, digoxigenin, glutathione, heparin, maltose, coenzyme A, protein A, Brilliant Blue FCF, azorubine, phytoestrogen, nickel, cobalt, zinc, poly-histidine, HA-tag, c-myc tag, FLAG-tag, S-tag, a hapten to an antibody, a mono- or oligosaccharide ligand to a lectin, hormones, cytokines, toxins, dyes, and vitamins.

In the context of the present disclosure, an “affinity capture domain” refers to a domain capable of binding a corresponding affinity domain. Example materials having such properties include avidin, streptavidin, neutravidin, maltose-binding protein, GST, antibodies, lectins, nickel, cobalt, zinc, and poly-histidine. Further examples include groups that form an irreversible bond with a protein tag, including benzylguanine or benzylchoropyrimidine (SNAP-tag); benzylcytosine (CLIP-tag); haloalkane (HaloTag); CoA analogues (MCP-tag and ACP-tag); trimpethoprim or methotrexate (TMP-tag); FlAsH or ReAsH (Tetracysteine tag); a substrate of biotin ligase; a substrate of phosphopantetheline transferase; and a substrate of lipoic acid ligase. An affinity group is used for selectively enriching samples by means of affinity purification methods, wherein the affinity binding partner is immobilized in a column, bead, microtiter plate, membrane or other solid support.

In the context of the present disclosure, “attach,” “fuse,” and “join” may be used interchangeably to refer to any means of linking two (or more) nucleic acid ends, for example, by ligases, transposases, topoisomerases, DNA-dependent protein kinases, and other end joining or repair enzymes, wherein the ends may be in the same molecule (e.g., to form a circular nucleic acid molecule from a linear substrate) or different molecules (e.g., to form a single linear nucleic acid product from two linear substrate molecules). Fusing two (or more) nucleic acid ends may comprise forming a covalent bond (e.g., a phosphodiester bond, a phosphorothioate bond, or any other linkage) between the ends. Fusion of a double-stranded nucleic acid may comprise fusing either or both 5′ and 3′ termini to either or both of the respective 3′ and 5′ termini of a second double-stranded nucleic acid. Fusion of a double-stranded nucleic acid may comprise fusing the 5′ and 3′ termini of a nick to one another. In some embodiments, fusing nucleic acid ends does not include nick translation.

In the context of the present disclosure, “coverage” refers to the number of times a targeted subset of a population of polynucleotides (e.g., fragmented genomic DNA) is sequenced.

In the context of the present disclosure, “enriched library” refers to a library having an increased abundance of one or more targets relative to a reference library. For example, an enriched cell-free fetal DNA library may have a higher abundance of one or more non-invasive prenatal testing targets than a maternal whole blood DNA library or a cell-free DNA library. The number of enriched targets in an enriched library may be 1, more than 1, more than 10, more than 100, more than 1,000, more than 10,000, more than 100,000, or more than 500,000. An enriched library may comprise enriched targets corresponding to an organism's entire exome.

In the context of the present disclosure, “linear amplification” refers to an amplification reaction in which the amount of product increases linearly, not exponentially, over time.

In the context of the present disclosure, “linear amplification annealing site” refers to a portion of a polynucleotide having a sequence that is (a) complementary to an amplification primer and (b) unique within the polynucleotide. A linear amplification annealing site may have sufficient complexity to be unique within a given polynucleotide in which it is present. For example, a linear amplification annealing site may be 15 nts, >20 nts, >25 nts, >30 nts, >35 nts, >40 nts, >45 nts, or >50 nts in length.

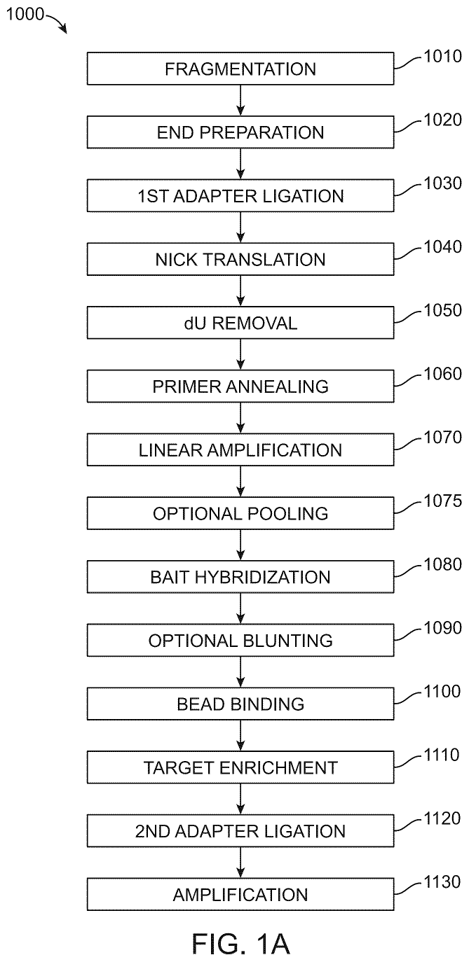

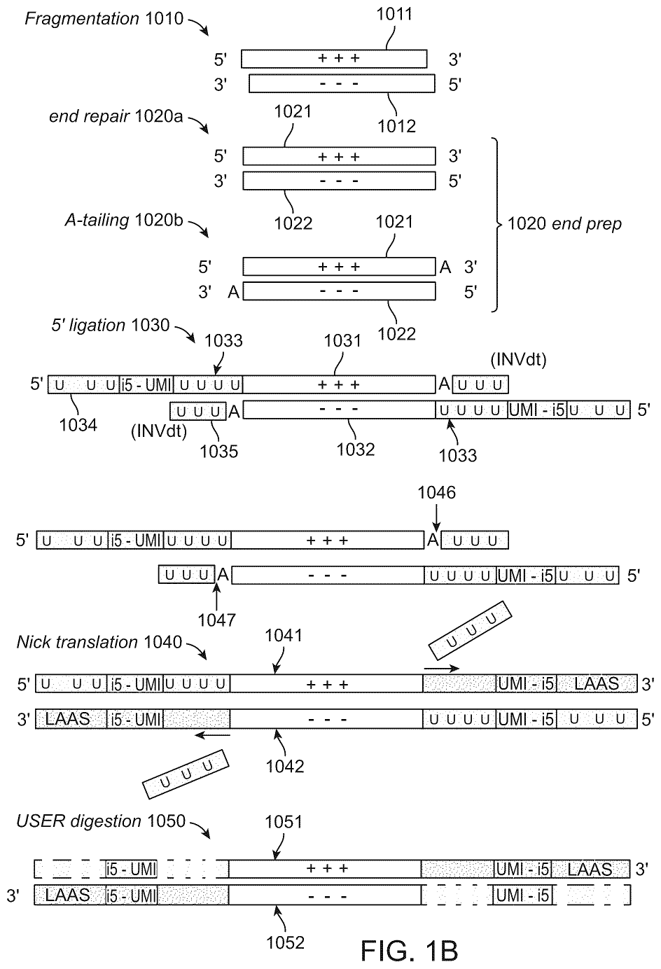

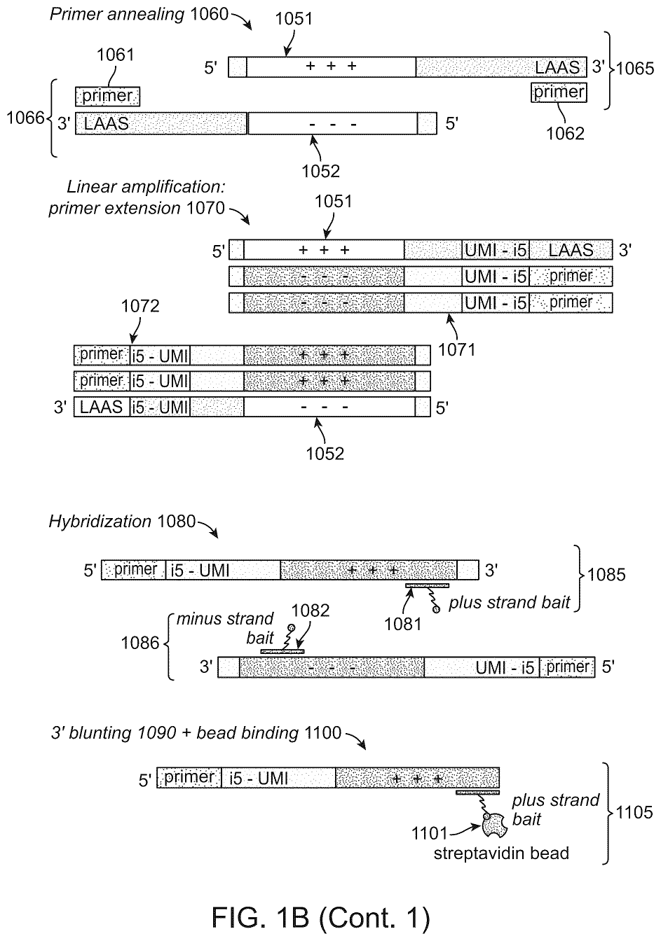

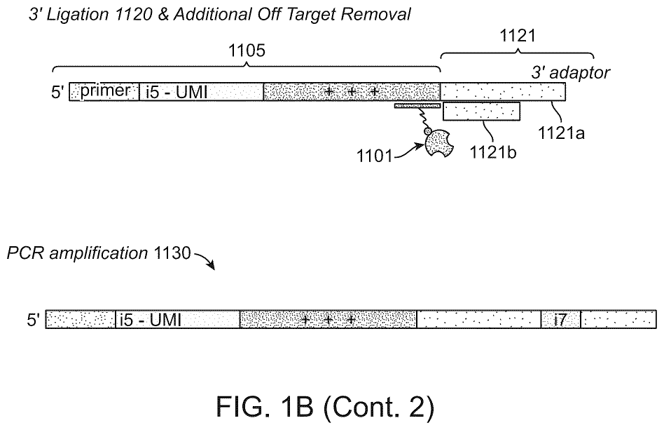

The term “high sensitivity” for sequencing reads refers to the detection of rare variants that may occur in genomes. For example, in cancer biopsies, only a small percentage e.g. 0.1% of a population of polynucleotides from a human sample may contain the sequence variant of interest (e.g. SNP). Therefore, a method that has a high sensitivity is necessary to detect these rare events. The methods involving linear amplification described herein and exemplified in FIGS. 1A-1B are high sensitivity methods.

In the context of the present disclosure, “non-naturally occurring” refers to a polynucleotide, polypeptide, carbohydrate, lipid, or composition that does not exist in nature. Such a polynucleotide, polypeptide, carbohydrate, lipid, or composition may differ from naturally occurring polynucleotides polypeptides, carbohydrates, lipids, or compositions in one or more respects. For example, a polymer (e.g., a polynucleotide, polypeptide, or carbohydrate) may differ in the kind and arrangement of the component building blocks (e.g., nucleotide sequence, amino acid sequence, or sugar molecules). A polymer may differ from a naturally occurring polymer with respect to the molecule(s) to which it is linked. For example, a “non-naturally occurring” protein may differ from naturally occurring proteins in its secondary, tertiary, or quaternary structure, by having a chemical bond (e.g., a covalent bond including a peptide bond, a phosphate bond, a disulfide bond, an ester bond, and ether bond, and others) to a polypeptide (e.g., a fusion protein), a lipid, a carbohydrate, or any other molecule. Similarly, a “non-naturally occurring” polynucleotide or nucleic acid may contain one or more other modifications (e.g., an added label or other moiety) to the 5′-end, the 3′ end, and/or between the 5′- and 3′-ends (e.g., methylation) of the nucleic acid. A “non-naturally occurring” composition may differ from naturally occurring compositions in one or more of the following respects: (a) having components that are not combined in nature, (b) having components in concentrations not found in nature, (c) omitting one or components otherwise found in naturally occurring compositions, (d) having a form not found in nature, e.g., dried, freeze dried, crystalline, aqueous, and (e) having one or more additional components beyond those found in nature (e.g., buffering agents, a detergent, a dye, a solvent or a preservative). All publications, patents, and patent applications mentioned in this specification are herein incorporated by reference to the same extent as if each individual publication, patent, or patent application was specifically and individually indicated to be incorporated by reference.

In the context of the present disclosure, “nick translation” of a double-stranded DNA molecule having at least one nick comprising a 3′-OH refers to templated addition of dNTPs beginning at the 3′-OH of the nick(s). Nick translation may also include 5′ to 3′ excision of nucleotides beginning from the nick(s). Nick translation may be catalyzed by DNA polymerases including, for example, E. coli DNA pol I, pol Ik, Taq DNA polymerase, OneTaq® DNA polymerase (New England Biolabs, Inc.), Bst DNA polymerase (e.g., large fragment), Bsu DNA polymerase, phi29 DNA polymerase, Therminator™ DNA Polymerase, Klenow, Vent® DNA polymerase, and Deep Vent® DNA polymerase. DNA polymerases with nick translation and/or strand displacement activity may be referred to herein as “nick translation polymerases”. A “nick” may comprise a break in the phosphate backbone with or without a short (e.g., up to 15 nucleotide) gap.

In the context of the present disclosure, “population of polynucleotides” or “library” refers to a composition comprising a plurality of polynucleotides having different properties (e.g., sequence, length). Each polynucleotide molecule in a population of polynucleotides may be unique relative to other polynucleotides in the population. Complex libraries may have more than 10⁴, 10⁵, 10⁶ or 10⁷ different nucleic acid molecules or may have fewer but longer molecules where the fewer molecules collectively have more than 10⁴, 10⁵, 10⁶ or 10⁷ nucleotides. Examples of complex populations of polynucleotides include mammalian and human DNA and cDNA libraries. A population of polynucleotides may comprise or may be derived from part or all of an organism's genomic DNA, organelle DNA, cDNA, mRNA, microRNA, long non-coding RNA, or other DNAs or RNAs. A population of polynucleotides may comprise or may be derived from artificial DNA or artificial RNA. A population of polynucleotides may be prepared, for example, by chemically, enzymatically, or physically fragmenting part or all of an organism's DNA or RNA to produce fragments having a size or sizes within a desired range. An enriched library may have a complexity of less than 10%, less than 5%, less than 1%, less than 0.5%, or less than 0.1%, less than 0.01%, less than 0.001%, less than 0.0001%, less than 0.00001%, less than 0.000001%, or less than 0.00000001% relative to a corresponding unenriched library.

In the context of the present disclosure, “polynucleotide” refers to any of a DNA, RNA, and mixtures thereof. A polynucleotide may be single stranded, double stranded, or partially single stranded and partially double stranded. A polynucleotide may have blunt ends, 5′ overhangs, and/or 3′ overhangs. A polynucleotide may be naturally occurring (e.g., purified or otherwise derived from an organism's genome (nuclear or organellar) or cytoplasm (tRNA, mRNA)) or synthetic. Polynucleotides may include an entire genome, a gene, a fragment of DNA or a library of fragments. A polynucleotide may have and/or may be depicted to have a plus and minus strand or a top strand and a bottom strand. Polynucleotides include a wide range of sizes (e.g., 2 nucleotides to 2 million nucleotides or even longer. Shorter polynucleotides (e.g., 2-250 nucleotides) may be referred to as polynucleotides or oligonucleotides.

In the context of the present disclosure, “sample” refers to a specific source of a specific population of polynucleotide fragments. Depending on its context, a sample may be a specific polynucleotide extraction from a single cell, a tissue or an individual biological entity such as a plant, animal or microbe.

In the context of the present disclosure, “sample tag” refers to a molecular barcode that identifies the sample source of a population of polynucleotide fragments. Accordingly, the adapters attached (e.g., ligated, joined or otherwise fused) at to each strand in a duplex will have the same sample tag as will other polynucleotide fragments in the population (e.g., FIG. 1B, i5 and i7 (Illumina)). A sample tag may be included in a first adapter, a second adapter, and/or an amplification primer (e.g., a PCR primer) prior to sequencing.

In the context of the present disclosure, an “strand” refers to a polynucleotide made up of nucleotides linked together by covalent bonds. Double-stranded or duplex polynucleotides (e.g., DNA) include two strands—a top or plus strand and a bottom or minus strand—each comprising sequences complementary to the other strand. Conventionally, illustrations of the strand designated as the top or plus strand is oriented with the 5′ end on the left and the 3′ end on the right and the strand designated as the bottom or minus strand is oriented with the 3′ end on the left and the 5′ end on the right.

In the context of the present disclosure, a “target sequence” refers to a portion of a polynucleotide fragment that constitutes or comprises a sequence of interest. A target sequence may be of interest for any of a variety of reasons, for example, because it is diagnostic of or otherwise associated with a disease, an infectious agent (e.g., a yeast, a bacteria, a virus, a phage), a phenotype, a genotype, an allele, a variation, or other state or condition. Examples of target sequences may include exons, introns, regulatory sequences, single nucleotide polymorphisms (SNPs), gene fusions, copy number variations, and indels. Analysis of target sequences may also be used to determine heterozygosity and homozygosity. A target sequence may be derived from sequential or nonsequential nucleotides in a genome. For example, a target sequence may constitute or comprise an expressed eukaryotic gene sequence (e.g., lacking some or all introns), a gene fusion (e.g., a fusion of two genes that may not occur naturally and/or may otherwise (normally) occur in separate regions of a genome), and/or an intron/exon boundary. A target sequence, for example, may constitute or comprise all or a portion of a gene selected from MTOR, DPYD, NRAS, NTRK1, DDR2, ALK, MSH2, MSH6, NFE2L2, IDH1, UGT1A1, VHL, RAF1, MLH1, CTNNB1, PIK3CA, FGFR3, PDGFRA, KIT, KDR, FBXW7, TERTpro, PIK3R1, APC, CSF1R, PDGFRB, FLT4, CCND3, ROS1, ESR1, PMS2, EGFR, CDK6, MET, SMO, BRAF, EZH2, FGFR1, JAK2, CD274, PDCD1LG2, CDKN2A, GNAQ, PTCH1, ABL1, TSC1, GATA3, RET, PTEN, FGFR2, CCND1, CCND2, KRAS, CDK4, FLT3, FLT1, BRCA2, RB1, AKT1, MAP2K1, IDH2, TSC2, TP53, ERBB2, BRCA1, RNF43, SMAD4, STK11, GNA11, MAP2K2, KEAP1, JAK3, AKT2, GNAS, NF2, ARAF, AR, BRIP1, CHEK2, PALB2, RAD51C, and RAD51D. In some embodiments, a target sequence may comprise all or part of an exon.

In the context of the present disclosure, a “target isolation probe” or “bait” refers to a molecule or complex comprising affinity domain and a target-specific oligonucleotide.

In the context of the present disclosure, a “target-specific oligonucleotide” refers to an oligonucleotide having a sequence complementary to at least a portion of a sequence of interest (a “target sequence”). Specificity of a target-specific oligonucleotide is conferred by its nucleotide selection. Specificity may be further influenced by the stringency of conditions under which it is contacted with another polynucleotide, for example, the temperature of contact and the absence or concentration salt(s), competitor(s), detergent(s), crowding agent(s) among others.

In the context of the present disclosure, “unique molecule identifier” (UMI) refers to a unique sequence of at least 6 nucleotides (6N) that together, with other like UMI's, have sufficient complexity to uniquely mark each polynucleotide molecule in a population of polynucleotide molecules. For example, the UMI of each molecule of adapter 1033 may have a unique sequence. UMI sequences may be random, haphazard, or otherwise varied to achieve a desired level of complexity. Longer random unique sequences may be used, for example, 2-15 nucleotides, 6-12 nucleotides, or 8-12 nucleotides.

The present disclosure provides methods for producing enriched libraries of target sequences. Methods according to some embodiments may efficiently produce libraries from low input samples that represent a large fraction (e.g., all or substantially all) of the nucleotides in the original samples.

### Sample Preparation

In some embodiments, a population of polynucleotides may be produced from any source or sample, for example, a sample of any organism, including, but not limited to, plants (e.g., ornamentals, vegetables, fruits, trees, woody shrubs, grasses, spore-bearing, seed bearing, and other plants), animals (e.g., vertebrates, invertebrates, reptiles, mammals, insects, worms, fish, birds, and other animals), bacteria, fungi (e.g., yeast), phage, viruses, cadaveric tissue, archaeological/ancient samples, any parts thereof (e.g., organs, tissues, cells, fluids, solids, biopsies), and any combinations thereof. In some embodiments, a sample for fragmentation may comprise genomic DNA from a mammal (e.g., a human or non-human animal). Mammalian bodily fluids of interest may include blood, serum, plasma, saliva, mucous, phlegm, cerebral spinal fluid, pleural fluid, tears, lactal duct fluid, lymph, sputum, synovial fluid, urine, amniotic fluid, and semen. In some embodiments, a sample may be obtained from a subject, e.g., a clinical specimen of a human subject. A sample may comprise, according to some embodiments, cell-free polynucleotides including, for example, circulating DNA of a bodily fluid (e.g., peripheral blood from the blood of a subject, for example, a cancer patient or a pregnant human) and DNA in the vascular fluids (e.g., phloem) of plants. In some embodiments, a sample for fragmentation may comprise genomic DNA from a plant (e.g., a leaf, a stem, a root, a seed, a pistil, a stamen, an embryo).

According to some embodiments, a method may include extracting polynucleotides (e.g., genomic DNA) from a biological source material and then fragmenting it mechanically (e.g., by sonication, nebulization, or shearing), enzymatically (e.g., using a double-stranded DNA “dsDNA” Fragmentase® enzyme (New England Biolabs, Ipswich MA), a transposase, a nuclease), or chemically (e.g., exposure to heat in the presence of divalent metal ions, formalin fixation and paraffin embedding). In some embodiments, a sample may comprise polynucleotides having a desired size or size range without further fragmentation, for example, where the population of polynucleotides is formed synthetically (e.g., in vitro) or collected from circulating cell-free DNA (cfDNA), e.g., ctDNA).

Populations of polynucleotide fragments, according to some embodiments, may have a median size that is below 1 kb. For example, a median or average size may be 50 bp to 500 bp or 80 bp to 400 bp, 150 bp-200 bp or 200 bp-300 bp or 300 bp-400 bp or 400 bp-500 bp or 500 bp-600 bp or 600 bp-700 bp). In some embodiments, populations of polynucleotide fragments may have a median size that is above 1 kb. For example, median fragment size may be 1 kb to 5 kb or 2 kb to 10 kb.

Methods of the present disclosure may be used, according to some embodiments, when the quantity of polynucleotides is limiting. For example, methods may be applied to populations of polynucleotides comprising or containing less than 200 ng of fragmented DNA (e.g., 10 μg to 200 ng of fragmented DNA, 100 μg to 200 ng of fragmented DNA, 1 ng to 200 ng of fragmented DNA, 5 ng to 50 ng of fragmented DNA) or less than 10,000 haploid genome equivalents (e.g., <5,000, <1,000, <500, <100, <10). Methods of the disclosure may also be used, according to some embodiments, when the quantity of polynucleotides is not so limiting.

### (First) Adapter Attachment

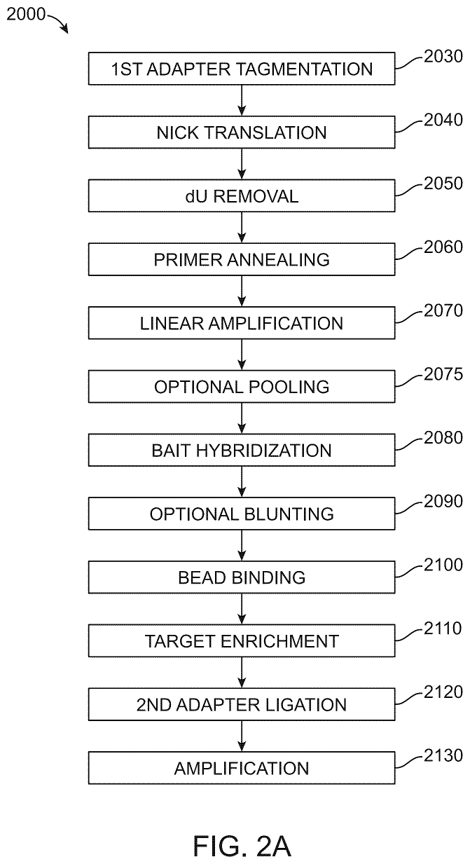

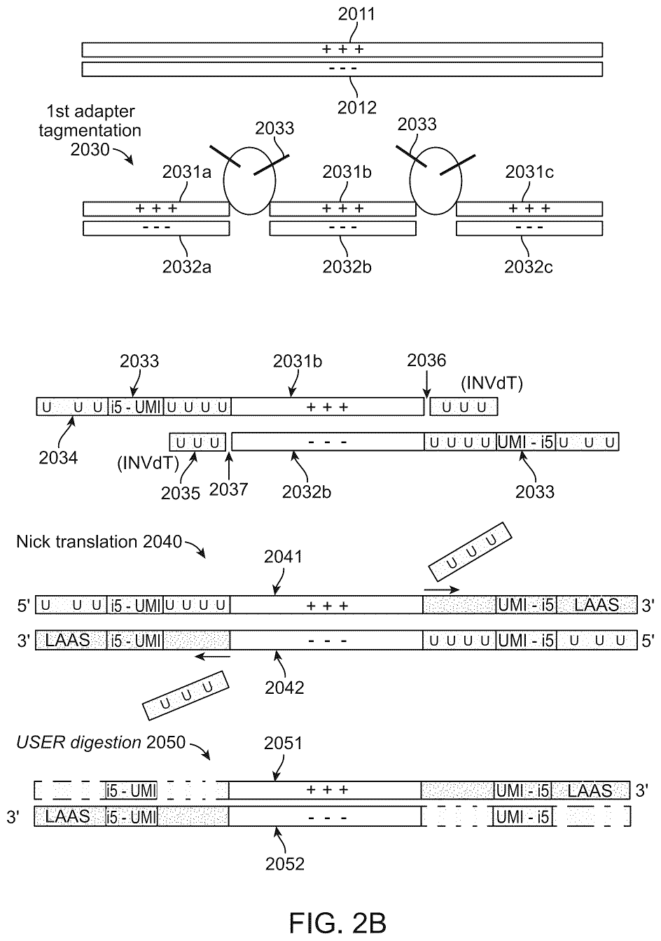

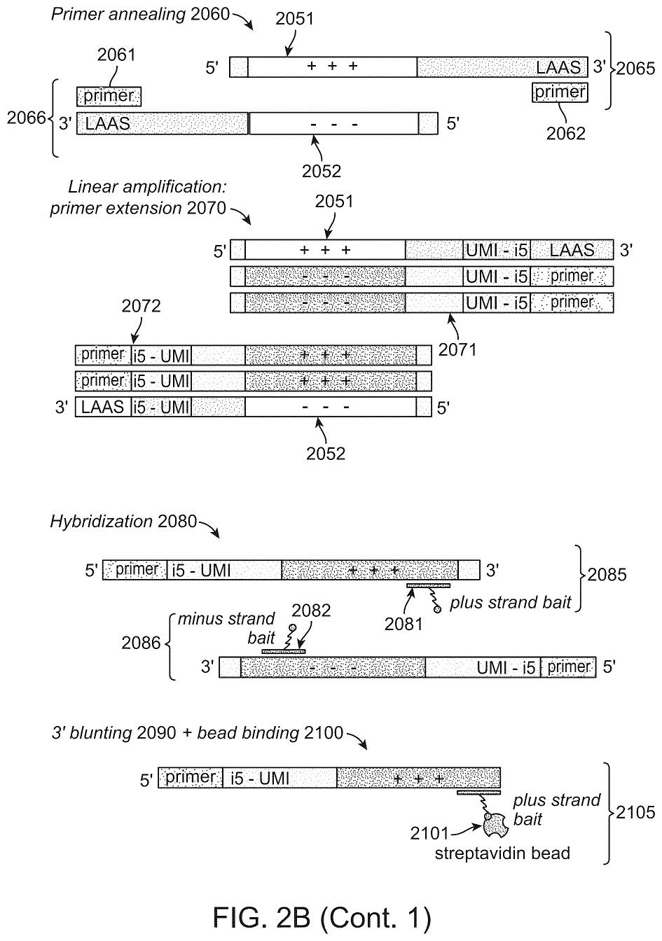

Methods may include, in some embodiments, attaching (e.g., ligating, joining or otherwise fusing) an adapter (e.g., a double-stranded adapter) to polynucleotide fragments to produce fusion products (tagged fragments). Adapters may comprise two separate strands hybridized together (e.g., as shown in FIG. 1B or 2B) or a single strand with portions hybridized to each other and an intervening single-stranded region (e.g., a hairpin or loop adapter). One or both strands of an adapter may comprise one or more (e.g., one, two or three) modified nucleotides, e.g., deoxyuridines, 8-oxoguanines or deoxyinosine. The presence of modified nucleotides may confer desirable properties on the adapter including, for example, limiting or preventing unwanted exonuclease digestion (e.g., phosphorothioates), reducing or eliminating unwanted secondary structure associated with G-rich regions (e.g., 8-aza-7-deazaG (“Super G”)), or increasing the melting temperature of duplexes (e.g., using locked nucleic acids or at specific A-T pairs by replacing the T with 5-hydroxybutynl-2′-deoxyuridine (“Super T)). In some cases the presence of modified nucleotides may afford a user an efficient option for removal of the adapter in a subsequent step, for example, by enzymatic removal (e.g., using a glycosylase) prior to linear amplification.

According to some embodiments, a method may include producing a population of A-tailed fragments from a population of polynucleotides (e.g., an unamplified, undenatured population of polynucleotides). A-tailing may include, for example, contacting a polynucleotide with a polymerase and dATP under conditions permitting or favoring the untemplated addition of a single dA to the 3′ end(s). Where fragments are A-tailed, adapters may have a corresponding 3′ overhang of a T or U to facilitate ligation. A-tailing polynucleotide fragments may facilitate ligation of adapters (e.g., through Watson-Crick base pairing of 3′ As and 5′ Ts or Us) which can be efficiently achieved using kits provided by New England Biolabs, Ipswich, MA (e.g. NEBNext® Ultra™ II End Repair/dA-Tailing Module). Alternatively, other means for attaching adapters to double-stranded DNA in a particular orientation may be used, or similarly A-tailing may be achieved by using kits from other vendors.

In some embodiments, an adapter comprising a top and bottom strand may be attached (e.g., ligated, joined or otherwise fused) to a polynucleotide fragmentation product, wherein the top strand of the adapter is attached (e.g., ligated, joined or otherwise fused) to the plus or minus strand of a fragmented polynucleotide (e.g., ligated to the 3′ end of an A-tailed fragmented polynucleotide). A top strand may comprise in a 5′ to 3′ direction, a sequence complementary to a linear amplification annealing site (e.g., an NGS platform-specific sequencing primer), optionally a sample tag, a unique molecular identifier (UMI), a sequence complementary to the bottom strand, and optionally a nucleotide complementary to A. In some embodiments, an adapter's top strand is longer than its bottom strand. A bottom strand of an adapter may have at least 10 nucleotides (e.g., at least 15 nucleotides). Shorter bottom strands may be appropriate under some conditions. The 5′ end of an adapter's bottom strand may comprise a 5′-OH, for example, to block ligation of the bottom strand to the bottom strand of a polynucleotide fragment. Adapter ligation may be performed by T4 DNA ligase, circligase, PBCV-1 DNA ligase, or another ligase. Ligation products optionally may be separated from other components of the ligation mixture.

In some embodiments, after ligation, a region of a hairpin or loop adapter can be cleaved to produce a duplex in which the top and bottom strands are on different molecules. In some cases, the cleaved region of a hairpin adapter may contain a modified residue such as deoxyuridine, and the base can be cleaved using a glycosylase (e.g., UDG), although other methods are known.

### Strand Displacement/Nick Translation

Fusion products (tagged fragments) comprising a nick (e.g., a nick defined by an unligated end of bottom strand of an adapter and the A-tail of the fragmented polynucleotide) may be contacted with a strand displacement polymerase or a nick translation polymerase for the templated addition of nucleotides. The products of either approach may be referred to as nick translation products. Nucleotides may be added, for example, beginning at the A-tail of the fragmented polynucleotide and the template is the top strand of the adapter. The nascent strand of the formed duplex polynucleotide may be complementary to the top strand of the adapter and optionally may be free of modified nucleotides (e.g., by performing strand displacement/nick translation in the absence of modified nucleotides). Each strand of the formed duplex polynucleotide may comprise, in a 5′ to 3′ direction, the top strand of the adapter, the polynucleotide fragment, and the nascent strand (which is complementary to the top strand of the adapter). Where the top strand of the adapter comprises one or more modified nucleotides, products of strand displacement/nick translation may be contacted with a single nucleotide excision reagent (e.g., a glycosylase and an endonuclease) to remove the modified nucleotides. For example, a single nucleotide excision reagent may remove the base of the modified nucleotide and cleave the phosphodiester backbone at the 3′ and 5′ sides of the modified base (or the abasic site resulting from removal of the modified base). For example, a top strand of an adapter comprising deoxyuridine may be contacted with UDG (e.g., Antarctic Thermolabile UDG (New England Biolabs, Ipswich, MA)), for example, at 37° C. for 10 minutes. UDG catalyzes the release of free uracil from the adapter sequences fused to the 5′ end of the DNA fragments and produces abasic sites in the adapter sequence. Abasic sites are susceptible to hydrolytic cleavage and break apart at the elevated reaction temperatures in the following thermocycling reaction. Alternatively, UDG may be used in combination with EndoVlll (e.g., USER®, New England Biolabs, Inc.). Other modified nucleotide/reagent combinations may be used. Sites of removed single nucleotides (e.g., deoxyuridine or other modified nucleotides) may define the ends of oligonucleotides of varying lengths depending, for example, on the location of the removed nucleotides in the initial strand, but in any case will be shorter than the initial strand. Consecutive modified nucleotides, when excised, may be referred to as single nucleotide gaps, consecutive single nucleotide gaps, or nucleotide gaps. Excising two modified nucleotides that flank an unmodified nucleotide may release that single nucleotide. Single nucleotides and oligonucleotides formed by the action of a single nucleotide excision reagent at working reaction temperatures (e.g., 100° C. to 40° C. (or higher)) may not remain base paired to the other strand. Accordingly, they may be lost in subsequent steps. In some embodiments, nucleotides and oligonucleotides formed by the action of a single nucleotide excision reagent may be removed/separated from the remaining strands comprising the polynucleotide fragments and the nascent strands from nick translation by optionally including a specific cleanup step.

### Linear Amplification

According to some embodiments, nick translation products (e.g., remaining after removal of the modified nucleotides) may be linearly amplified. Linear amplification may be performed by contacting fusion products (e.g., concurrently with or following removal of modified nucleotides) with a polymerase, dNTPs, and a linear amplification primer to produce a reaction mix. In some embodiments, a polymerase may be a thermostable polymerase and a reaction mix may be thermocycled at least once (e.g., at least 5 times, or at least 10 times or at least 20 times) to produce a number of copies of each tagged fragment where the copy number corresponds to the cycle number. Linear amplification may be performed using NEBNext Ultra II Q5© Master Mix (A master mix containing Q5 DNA polymerase (New England Biolabs, Ipswich, MA)), although other polymerases can be used. Products of linear amplification may have a copy of the molecular barcode of the top strand of the adapter on the 3′ side of the polynucleotide fragment and may be referred to as 5′-tagged amplification products.

In some embodiments, a polymerase active at lower temperatures may be used (e.g., Klenow exo-, Bsu large fragment, and phi29 for moderate temperature reactions (25-40° C.) and the large fragment of Bst DNA polymerase for higher temperature (50-65° C.) reactions) and a reaction mix may be incubated with or without thermocycling to produce a number of copies of each tagged fragment. For example, linear amplification may be performed using isothermal amplification (e.g., instead of thermocycling). Examples of isothermal amplification may include ligase chain reaction (LCR), strand displacement amplification (SDA), transcription mediated amplification (TMA), self-sustained sequence replication (3SR), QP replicase based amplification or rolling circle amplification, nucleic acid sequence-based amplification (NASBA), repair chain reaction (RCR), boomerang DNA amplification (BDA) helicase dependent amplification (HDA).

Linear amplification may be performed, according to some embodiments, by denaturing the polynucleotides to be amplified (e.g., at 98° C. for 30 seconds), contacting the denatured polynucleotides with a polymerase, dNTPs, and a linear amplification primer to produce a reaction mix, and thermocycling the reaction mix at least once (e.g., at least 5 times, at least 10 times, at least 15 times or at least 20 times). Thermocycling may be performed with the following steps: (1) a temperature above 90° C. (e.g., 98° C.) for at least 5 seconds, (2) a temperature of below 60° C. (e.g., 55° C.) for at least 5 seconds, and (3) a temperature in the range of 65° C. to 80° C. (e.g., 70° C. to 75° C.) for at least 10 seconds. At the first temperature, polynucleotide fragments denature. At the next temperature, the linear amplification primer anneals to the 3′ end of the adapter sequence. At the third temperature, the polymerase (e.g., Q5 polymerase) extends the linear amplification primer. Other thermocycling conditions are known and may be readily used.

It may be desirable, in some embodiments, to use a polymerase for linear amplification with a low error rate (e.g., 4× to 8× fewer errors than Taq polymerase). Linear amplification may be performed with a proofreading DNA polymerase, which may have a 3′ to 5′ exonuclease activity. Examples of proofreading thermostable polymerases include Pfu (Agilent Technologies, Santa Clara, CA), Pwo (Roche, Basel, Switzerland), Tgo (Roche, Basel Switzerland), VENT® (New England Biolabs, Ipswich, MA), DEEP VENT® (New England Biolabs, Ipswich, MA), KOD HiFi (Novagen, Madison, WI), PFX50™ (Invitrogen, Waltham, MA), HERCULASE II™ (Agilent Technologies, Santa Clara, CA), PLATINUM PFX™ (Life Technologies, Waltham, MA) and ProofStart™ (Qiagen, Hilden, Germany). Further examples of proofreading thermostable polymerases include, but are not limited to, PHUSION® (Thermo Fisher Scientific, Waltham, MA), PFUULTRA™ (Agilent Technologies, Santa Clara, CA), PFUULTRA™ II (Agilent Technologies, Santa Clara, CA), PROOF™ (Bio-Rad, Hercules, CA), Q5 polymerase, and KAPAHIFI™ (Kapa Biosystems, Wilmington, MA). These polymerases may produce, on average, at least 20× fewer errors than Taq polymerase and can be readily employed herein. Linear amplification may be performed with a non-proofreading DNA polymerase. Examples of non-proofreading thermostable polymerases (i.e., thermostable polymerases that do not have a 3′ to 5′ exonuclease activity) include, but are not limited to, Taq and Tth.

In some embodiments, linear amplification reduces or avoids amplification bias and/or amplification of sequence errors (e.g., nucleotide incorporation errors that may occur in early rounds of amplification). Linear amplification may provide a more streamlined process (e.g., relative to non-linear amplification) by avoiding the need for additional steps or measures to safeguard product quality. For example, non-linear amplification methods may omit the USER® digestion step to retain adapters on both ends of the fragments, but would then benefit from including blocking oligonucleotides to retain specificity of the subsequent bait hybridization. Such oligos would be needed to prevent a bait hybridizing to a target fragment and that target fragment's adaptor sequence hybridizing to the complementary adaptor sequence of a random non-target fragment. Such hybridization may lead to formation of “daisy chain” amplification products comprising target and off target molecules. Nevertheless, non-linear amplification may be an option in some embodiments, for example, where a loss in quality may be acceptable and/or where additional precautions are included to off-set a loss in quality.

### Hybridization

Linear amplification products (e.g., 5′-tagged amplification products) may be contacted with (e.g., hybridized to) target-specific oligonucleotides to produce complexes. In some embodiments, target isolation probes may comprise target-specific oligonucleotides linked (e.g., covalently) to an affinity domain, for example, biotin. Target-specific oligonucleotides may be designed to hybridize to any target including targets of diagnostic, and/or therapeutic interest (e.g., target sequences in cancer-related genes) and targets of breeding interest (e.g., traits, genotypes, phenotypes and markers thereof). Contacting linear amplification products with target isolation probes may include contacting the linear amplification products with target isolation probes having at least 1, at least 5, at least 10, at least 100, at least 1,000 or at least 10,000 target-specific oligonucleotides, each with a different specificity, to isolate and enrich fragments having different elements of a target region or genome. Each target-specific oligonucleotide may have a length of 30 to 100 nucleotides. Shorter and longer target-specific oligonucleotides may be used under some conditions/circumstances. For example, smaller targets may be desired where the polynucleotide fragments to be evaluated are small, such as fragments from arising from urine cfDNA. Smaller targets may also be desired or acceptable when evaluating small genomes (where shorter sequences retain uniqueness/specificity). In some embodiments, target-specific oligonucleotides pairs may be used, with one member of the pair hybridizing to a top strand of a target and the other member of the pair hybridizing to a bottom strand of the target.

A target sequence may lie at the 3′ end of a region of interest. In some embodiments, biotinylated target-specific oligonucleotides are added to a hybridization solution containing the pool of DNA fragments. The hybridization solution containing the target-specific oligonucleotides and DNA fragments is then incubated to allow the target-specific oligonucleotides to hybridize 5′ tagged amplification products that comprise a target sequence. This hybridization may be performed at relatively high stringency or relatively low stringency. If low stringency conditions are used, a significant amount of non-specific binding to the target-specific oligonucleotides may occur. Non-specifically bound sequences can be removed by treatment with a 3′-5′ single-stranded exonuclease and a double-stranded exonuclease (e.g., exonuclease Ill) in a subsequent step.

Complexes (e.g., comprising target isolation probes hybridized to a target region of a tagged fragment), in some embodiments, may be captured on a support by an affinity capture domain (e.g., streptavidin) linked (e.g., covalently) to the support. The tagged fragments comprising the target may be thereby enriched or separated from tagged fragments without the target. In some embodiments, magnetic beads coated in streptavidin may be added to the reaction mix after hybridization of the 5′ tagged amplification products to the target-specific oligonucleotides. The magnetic beads can be isolated by magnetism and then washed, thereby enriching for complexes that comprise the 5′ tagged amplification products. An alternative to biotin includes a SNAP-Tag® (New England Biolabs, Ipswich, MA) that is a protein that reacts with a benzylguanine and may be modified to bind to an affinity capture domain.

A solid support may include a matrix formed from the capture domain or coated with the capture domain. A solid support may be, for example, a bead including a magnetic bead, a column, a porous matrix, or a flat surface formed from for example, plastic or paper.

### Producing 3′ Blunt Ends

Affinity captured complexes, in some embodiments, may have 3′ overhangs relative to the 5′ terminus of the hybridized target-specific oligonucleotide. A 3′ overhang may comprise one or more 3′ nucleotides of a fragmented polynucleotide and/or one or more nucleotides corresponding to the top strand of the adapter. A 3′ overhang may be removed by contacting affinity captured complexes with a single-stranded 3′-5′ exonuclease to produce blunt ends, flush with the 3′ end of the target sequence. One or more single stranded 3′-5′ exonucleases may be used individually or in combination to catalyze stepwise removal of mononucleotides from 3′ ends of single-stranded DNA to form a flush end. For example, exonuclease I and exonuclease T can be used individually or in combination. A 3′-5′ single strand exonuclease may trim the 3′ end of the captured 5′-tagged amplification products until it is flush with the target sequence. A flush end may comprise a 5′ overhang (e.g., a 1-5 nucleotide 5′ overhang), for example, where a 3′-5′ single strand exonuclease trims the 3′ end of the captured 5′-tagged amplification products a little beyond the target sequence. A 3′ nuclease reaction buffer containing one or more single-stranded 3′ exonucleases, in some embodiments, may be added to captured complexes (which still include 5′ tagged amplification products) to create a reaction mix. After the reaction mix has been incubated (e.g., for approximately 10 minutes at 25° C., and then 5 minutes at 37° C.), captured complexes may be separated from the remaining mix (e.g., by magnetism in the case of affinity capture domains bound to magnetic beads or by decanting, pipetting, or otherwise removing liquid where affinity capture domains are bound to a surface of a tube or plate) and washed. The mix can then be discarded because the exonuclease-treated complexes are still tethered to the support (e.g., beads, tube, plate).

### (Second) Adapter Fusion

According to some embodiments, a 3′ adapter (e.g., double-stranded adapters) may be attached (e.g., ligated, joined or otherwise fused) to the 3′ end of captured polynucleotide fragments of exonuclease-treated complexes to produce strands of 3′-tagged polynucleotide fragments comprising the target sequence, which polynucleotide fragments may also remain tagged at their 5′ ends from the preceding (first) adapter fusion. For example, captured complexes (e.g., following washing) may be contacted with a 3′adapter, a ligase and/or a ligation buffer and incubated for an appropriate time and appropriate temperature to permit ligation. In some embodiments, a 3′ adapter may comprise a blocking moiety at its 3′ end(s), for example, where it may be desirable for the resulting ligation products to be resistant to 3′-5′ double-stranded DNA exonucleases. Blocking moieties may include one or more phosphorothioates at the 3′ end, having a single-stranded portion at the 3′ end or having a hairpin structure, which becomes single-stranded after it has been partially digested. In some embodiments, a 3′ adapter may have a long (e.g., >3 nt, >4 nt, >5 nt, >10 nt, >20 nt, >25 nt, >50 nt, or 100 nt) 3′ single strand overhang making it resistant to digestion by double stranded exonucleases.

As an example, a 3′ adapter sequence and T4 DNA ligase may be added to the ligation mixture to blunt end ligate the 3′ adapter to the target sequence. A-tailing the 3′ end of the target sequence for ligation with an adapter having a complementary T overhang may be an alternate. Other ligation methods known in the art may be used including, for example, joining the 3′ adapter sequence to the target sequence by ligation using for example, a circ ligase or Taq ligase. In this example, the 3′ adapter is double-stranded with its top strand ligating to the 3′ end of the polynucleotide sequence fragments, and its bottom strand containing several deoxyuridine residues or other modified nucleotides, as described above. A plus strand of a 3′ adapter may comprise a 3′ adapter sequence (e.g., NGS platform-specific sequencing primer site and a library amplification primer site). After incubation, e.g., at 20° C. for 15 minutes, ligation products (e.g., complexes with ligated 3′ adapters still bound to the affinity capture domain and support) may be isolated from the ligation mixture (e.g., using magnetism). The separated ligation mix (now lacking the ligation products) may be discarded since the ligation products, which comprise a complex comprising a 5′ and 3′ tagged strand comprising the target sequence, remain tethered to the support.

### Sample Clean-Up and Amplification

In some embodiments, second adapter fusion products, which include strands comprising the target sequence and tagged at both 5′ and 3′ ends, may be cleaned up. For example, strands comprising the target sequence and tagged at both 5′ and 3′ ends may be separated or isolated from unligated target isolation probes, target isolation probes ligated to the lower strand of the 3′ adapter, off-target polynucleotides (e.g., polynucleotides lacking the target sequence) by contacting second adapter ligation products with a double-stranded 3′-5′ exonuclease that catalyzes the stepwise removal of mononucleotides from 3′ ends of duplex DNA, and also cleaves abasic sites. A double-stranded 3′-5′ exonuclease may eliminate any remaining double-stranded off-target DNA molecules.

As an example, magnetic beads may be washed and resuspended in 3′-double-stranded exonuclease buffer. A double-stranded 3′-5′ exonuclease, e.g., exonuclease Ill, may be added to form a mixture and the exonuclease mixture may be incubated (e.g., 15 minutes at 37° C.) thereby removing any remaining non-target sequences with unprotected or accessible double-stranded 3′ ends. UDG or another deglycosylase can also be added to remove the bases from the modified nucleotides (e.g., the deoxyuridines) from the 3′ adapter minus strand. In this example, target-specific oligonucleotides are permitted to remain hybridized to target sequences and magnetic beads may be isolated from the remaining reaction mix using magnetism and washed. The remaining reaction mix may be discarded since the exonuclease-treated ligation products, which comprise a complex comprising a 5′ and 3′ tagged strand comprising the target sequence, are still tethered to the beads. Tagged strands may be rendered 3′-5′ exonuclease insensitive to protect them from degradation during this exonuclease treatment.

Optionally, strands comprising the target sequence and tagged at both 5′ and 3′ ends may be amplified by PCR, for example, by contacting the strands with a first primer that hybridizes with a portion of the 3′ adapter and a second primer that hybridizes to the linear amplification primer sequence of the first adapter to produce PCR products. Contacting may further comprise contacting the strands with a PCR mixture comprising water and a PCR master mix (e.g., a master mix comprising a polymerase, dNTPs, metal ions, salts, and/or a buffer. PCR reaction conditions may include (a) a temperature above 90° C. (e.g., 98° C.) for at least 30 seconds, (b) 18 cycles of (1) a temperature above 90° C. (e.g., 98° C.) for at least 10 seconds, (2) a temperature in the range of 60° C. to 70° C. (e.g., 62° C.) for at least 15 seconds, and (3) a temperature higher than step (2) (e.g., 72° C.) for at least 20 seconds, and (c) a temperature of 72° C. for at least 3 minutes. According to some embodiments, strands comprising the target sequence and tagged at both 5′ and 3′ ends are not amplified.

### Sequencing

Strands comprising the target sequence and tagged (e.g., with appropriate sequencing compatible sequences) at both 5′ and 3′ ends, whether or not amplified, may be sequenced (e.g., by any desired sequencing method) according to some embodiments. For example, strands may be sequenced by a next generation sequencing method resulting in at least 10,000, at least 50,000, at least 100,000, at least 500,000, at least 1M at least 10M at least 100M or at least 1B sequence reads using suitable primers (e.g., primer extension using Illumina's reversible terminator method, Roche's pyrosequencing method (454), Life Technologies' sequencing by ligation (the SOLiD platform), Life Technologies' Ion Torrent platform, MGI's CoolMPS, Oxford Nanopore sequencing, and Pacific Biosciences' fluorescent base-cleavage method, among others). Reads may be paired end reads. Sequences needed for a selected sequencing method may be included in any desired adapter and/or primer including the second adapter, the PCR primer used after ligation of the second adapter, or both.

Examples of sequencing methods are described in the following references: Margulies et al. (Nature 2005 437: 376-80); Ronaghi et al. (Analytical Biochemistry 1996 242: 84-9); Shendure (Science 2005 309: 1728); Imelfort et al. (Brief Bioinform. 2009 10:609-18); Fox et al. (Methods Mol Biol. 2009; 553:79-108); Appleby et al. (Methods Mol Biol. 2009; 513:19-39); English (PLoS One. 2012 7: e47768); and Morozova (Genomics. 2008 92:255-64), which are incorporated by reference for the general descriptions of the methods and the particular steps of the methods, including all starting products, reagents, and final products for each of the steps. Sequence reads may be analyzed computationally to identify sequence variations in the sample, such as point mutations, deletions, insertions and rearrangements.

### Multiplexing

Advantages of multiplexing include (a) the ability to analyze a large number of samples in one sequence reaction while maintaining a means to track the source of each polynucleotide and each sample from which it came (b) pooling samples can increase efficiency and reduce cost of the workflow used to enrich targets and sequence samples. Multiplexing as described herein may involve pooling two, tens, hundreds or thousands of samples.

In some embodiments, the adapter fused onto the fragments in the initial step of the method may have a sample tag and the reaction may be multiplexed by combining samples from different sources (e.g., each with its own tag) into a pool. A pool may be distinguished (e.g., from other samples or pools) by its sample tag. In some embodiments, a method may comprise attaching (e.g., ligating, joining or otherwise fusing) a double-stranded adapter to individual polynucleotide fragments from a single sample having a first sample tag and then pooling the fused polynucleotide fragments with polynucleotides having one or more other sample tags from one or more other samples. If a sample is subjected to linear amplification, then an adapter with top strand comprising in a 5′ to 3′ direction, a sequence complementary to a linear amplification annealing site, optionally a sample tag, a UMI, a sequence complementary to the bottom strand, and optionally a nucleotide complementary to A, may be attached (e.g., ligated, joined or otherwise fused) to the 5′ ends of the sample DNA. During linear amplification, primer extension produces 5′ tagged amplification products. Because this reaction may be cycled multiple times and create many copies of the tagged polynucleotide fragment, this strategy is useful for high depth of coverage sequencing needs, such as analysis of cfDNA. Alternatively, if a sample is not subjected to linear amplification, then an adapter sequence having a sample tag, and optionally a UMI may be attached (e.g., ligated, joined or otherwise fused) to the 5′ end of the sample DNA. This strategy is suitable for low-depth of coverage sequencing needs of samples that are not limiting, such as genotyping.

Sample preparation, fragmentation, repair, and A-tailing steps may be performed as described herein using multiple samples in parallel in some multiplexing embodiments. A double-stranded adapter may comprise a bottom strand and a longer top strand that comprises a sample tag positioned on the single strand extension located between a first single stranded adapter sequence and the duplex region that contains a sequence that is complementary to the bottom strand. Each sample may be prepared to include a distinct sample tag, where each tagged polynucleotide fragment may be traced back to the sample from which it came by its sample tag.

As shown, after all the samples have been attached to adapters that have a sample tag and optionally a UMI sequence, the samples may be pooled in a single vessel and may progress through the rest of the enrichment steps together. Optionally, samples may be pooled after linear amplification. After pooling, the method may comprise hybridizing the pool of adapter-tagged samples with a target-specific oligonucleotide to produce complexes. The complexes may be bound to a solid support, thereby enriching for 5′ tagged molecules that comprise a target sequence. If the target-specific oligonucleotides are biotinylated, then the enrichment may be performed using a support that comprise streptavidin (e.g., magnetic streptavidin beads). Next, in some embodiments, a method may comprise treating complexes with a 3′-5′ single strand exonuclease to remove any overhanging 3′ ends from the product molecules and produce a flush end at the 3′ end of the target sequence. However, this step is optional and a second adapter may be added by another means. A second double-stranded adapter may be attached (e.g., ligated, joined or otherwise fused) to the flush end of the exonuclease-treated complexes, thereby adding a 3′ adapter sequence onto the 3′ end of the target sequence to produce a 5′ and 3′ tagged strand comprising the target sequence. Other fusion strategies may be used in this step. For example, the exonuclease-treated complexes may be A-tailed and a T-tailed adapter could be used. A strand (e.g., a top strand) of a second double-stranded adapter may be 3′-5′ double-stranded DNA exonuclease resistant because of a 3′ blocking moiety. In these embodiments, the method may comprise treating the ligation products with a 3′-5′ double-stranded exonuclease. The 5′ and 3′ tagged strands may then be amplified by PCR using a first primer that hybridizes to the 3′ adapter sequence and a second primer that hybridizes to the complement of the first adapter sequence, to produce amplification products. In this step, a sample tag may be added to identify the pool of samples in the multiplex reaction.

Amplification products may be sequenced by any convenient method to produce sequence reads that comprise the sequence, at least part of the target sequence and a sample tag or complements thereof. During analysis, the sequence reads may be assigned to a sample on the basis of the sample tag that is in the sequence read. This method may be implemented in a high-throughput format. As few as 1 and as many as 96 samples, or as many as 384 or more samples, each having different sample tags, may be pooled together where the pool is labeled with a single sample tag on the 3′ adapter. These pooled samples each with a single sample tag may then be pooled into larger pools containing multiple sample tag sequences for analysis in a single sequencing reaction. A single sequencing reaction may include a multiplex enriched preparation of 3′ adapter and 5′ adapter fused polynucleotide target sequences from one or more samples, 2 or more samples, 3 or more samples, 5 or more samples, 10 or more samples, 50 or more samples, 100 or more samples, 500 or more samples, 1000 or more samples, 5000 or more samples, up to and including about 10,000 or more samples where these samples may be obtained from the same or different sources. For example, samples may be plant extracts, and the sources may be different plants. In this example, the original individual polynucleotide fragments containing a target sequence may be tracked by a UMI, each from which the polynucleotides came, may be tracked by a sample tag and each plant for which the seeds came may be tracked by an sample tag sequence.

The foregoing example may include further multiplexing in connection with the hybridization. Hybridization reactions may be performed with single or pairs of target isolation probes or they may be multiplexed by performing each reaction with many hybridization probes (e.g., 3 or more, 5 or more, 10 or more, 100 or more, or 1,000 or more target isolation probes).

### Kits

The present disclosure relates to kits for performing methods described herein. A kit, for example, may include any system for delivering materials or reagents for carrying out a method described herein. In some embodiments, kits may include systems that allow for the storage, transport, or delivery of reaction reagents (e.g., probes, enzymes, adapters, primers, reaction reagents, reaction vessels and/or surfaces in appropriate containers) and/or supporting materials (e.g., written instructions for performing the assay, handling instructions) from one location to another. For example, in some embodiments kits include one or more enclosures (e.g., boxes) containing one or more reaction reagents and/or supporting materials. Such contents may be delivered to the intended recipient together or separately. For example, a first container may contain an enzyme for use in an enrichment method, while a second container may contain adapters. A kit alone or in combination may be formulated for selecting and enriching target templates from a nucleic acid sample containing non-target and target sequences. A kit may include one or more adapters as described herein, primers; exonucleases; ligase; polymerase(s); buffers; and nucleotides. A kit may further comprise one or more buffer solutions and standard solutions for the creation of a DNA library. These components may be present in a single reaction vessel or multiple tubes and may be packaged separately or together.

### Automated Work Flows

Methods disclosed herein, may be performed with at least some automation. Systems for processing multiple samples in parallel may be adapted for use with the disclosed methods. For example, systems for processing samples in racks of tubes, multi-well plates, on droplets on surfaces, and/or through microfluidics (including variations that use pressure, electrical potential, acoustic forces, and/or other forces to manipulate fluids and contact materials). Methods disclosed herein may be performed, for example, using an Echo® 525 Liquid Handler (Labcyte, Inc., San Jose, CA) or by means of microfluidic devices or a lab on a chip (Aqua Drop, Sharp). For the methods shown in the FIG. 1A, steps 1010, 1020, and 1030 may be performed in a single buffer which then is replaced by a different buffer in step 1040. Steps 1050-1090 may be performed in a single reaction tube by adding reagents sequentially or together. In some embodiments, steps from sample to sequencing may be automated on a single device.

In some embodiments, it may be desirable to combine an enriched library with one or more other enriched libraries, for example, for sequencing. For example, multiple environmental specimens may be collected and enriched libraries (e.g., enriched for possible pathogenic DNA present) prepared from each. This may be achieved in a platform that utilizes, for example, 96 well dishes where 5′ adapter addition, hybridization, capture, enrichment and 3′ adapter addition is performed in individual wells of 96-well plates. Following 5′ adapter fusion of polynucleotide fragments from a single sample (a single environmental specimen) is achieved, polynucleotide fragments from multiple samples (specimens from multiple locations or from a single location over time) from all 96 wells may be combined into a single well in a second 96 well plate for capture enrichment and 3′ adapter fusion. Multiplexed samples from a plurality of wells in the second plate (representing multiple specimens) may then be pooled for sequencing. Sequence data may be deconvoluted using sample tags and/or UMIs to associate specific sequences with specific specimens.

### Examples

Some specific example embodiments may be illustrated by one or more of the examples provided herein.

### Example 1: Library Preparation Including Fragmentation and Adapter Ligation

In some embodiments, enrichment methods may be practiced in accordance with the example shown in FIG. 1A and FIG. 1B. As illustrated, a method 1000 may comprise fragmenting 1010 polynucleotides (e.g., genomic DNA) to form fragmented polynucleotides. Fragmented polynucleotides may comprise hybridized plus 1011 and minus 1012 strands. Fragmentation 1010 may comprise physically, mechanically, chemically, and/or enzymatically fragmenting the polynucleotides.

In some embodiments, a method may comprise preparing 1020 the ends of the fragmented polynucleotides for adapter ligation. Preparing 1020 ends of fragmented polynucleotides for adapter ligation may comprise (a) repairing 1020a fragmented polynucleotide ends to form blunt 5′ ends and blunt 3′ ends, (b) A-tailing 1020b the 3′ ends, or (c) both (a) and (b) (e.g., in sequence) to form single-base A overhangs at each 3′ end. The prepared fragmented polynucleotides comprise plus 1021 and minus 1022 strands hybridized to each other and comprise blunt ends or A-tailed ends.

A method may comprise contacting 1030 (e.g., ligating) fragmented polynucleotides having A-tailed 3′ ends with a first adapter (e.g., double-stranded adapter, a single-stranded hairpin adapter) having a blunt end or a 5′ nucleotide complementary to the 3′ A tail. In some embodiments, a first adapter, adapter 1033, may comprise a top strand 1034 and a bottom strand 1035. A double-stranded adapter, in some embodiments, may comprise a plurality of deoxyuridines in the top strand, in the bottom strand or in both the top and bottom strands. A top strand 1034 of adapter 1033 may comprising, in a 5′ to 3′ direction, a sequence complementary to a linear amplification annealing site (“LAAS” in FIG. 1B), optionally a sample tag (e.g., i5), a unique molecular identifier (UMI), a sequence complementary to the bottom strand 1034, and a nucleotide complementary to A. A bottom strand 1035 of adapter 1033 may comprise, in a 5′ to 3′ direction, a 5′-OH (e.g., a 5′ terminus lacking a phosphate), a sequence complementary to the top strand and a non-extendible modified nucleotide (e.g., an inverted dT). Contacting 1030, in some embodiments, may comprise ligating the 3′ end of the adapter top strand to the 5′ end of the fragmented polynucleotides without ligating the 5′ end of the adapter bottom strand to the 3′ (A-tailed) end of the fragmented polynucleotides to form double-stranded ligation products comprising a plus strand 1031 comprising a single-stranded break or nick and a minus strand 1032 comprising a single-stranded break or nick.

According to some embodiments, a method 1000 may comprise contacting 1040 double-stranded ligation products comprising nicks 1046 and 1047, with a nick-translating or strand displacing polymerase to form a nick-translated duplex polynucleotide comprising a plus strand 1041 and a minus strand 1042. Examples of nick translating polymerases include E. coli DNA polymerase I, pol Ik, Taq DNA polymerase, OneTaq® DNA polymerase (New England Biolabs, Inc.), Bst DNA polymerase (e.g., large fragment), Bsu DNA polymerase, phi29 DNA polymerase, Therminator™ DNA Polymerase, Klenow, Vent® DNA polymerase, and Deep Vent® DNA polymerase.

A method 1000 may include, in some embodiments, contacting 1050 the nick-translated duplex polynucleotide (e.g., comprising one or more modified nucleotides) with a single nucleotide excision reagent to form polynucleotide duplexes comprising a plus strand 1051 and a minus strand 1052, each comprising gaps (e.g., previously occupied by modified nucleotides) defining oligonucleotides of varying (e.g., generally short) lengths. For example, a plus strand 1051 of formed polynucleotide duplexes may comprise, in a 5′ to 3′ direction, a portion having gaps (e.g., single nucleotide previously occupied by U), a plus strand of the double-stranded fragmented polynucleotide, and a portion comprising a sequence complementary to the first adapter, a UMI, a sample tag, and a linear amplification annealing site.

Examples of a single nucleotide excision reagent may comprise an enzyme preparation that achieves uracil removal and phosphodiester bond cleavage (e.g., a USER® enzyme M5505 or M5508 (New England Biolabs, Inc.)) or may comprise a combination of enzymes (e.g., uracil DNA glycosylase in combination with endonuclease VIII. It will be appreciated that some polynucleotide sequences may comprise deoxyuridines at consecutive sequences. Treatment with a single nucleotide excision reagent may result in corresponding consecutive single nucleotide gaps, that is gaps of 2 or more nucleotides, according to the number of consecutive deoxyuridines originally present. A method may optionally include removal of the oligonucleotides formed by the treatment with the single nucleotide excision reagent.

A method 1000 may comprise contacting 1060 each strand (1051, 1052) of tagged fragments (polynucleotide duplexes comprising double-stranded fragmented polynucleotides and 3′ overhangs (e.g., 3′ overhangs comprising, in a 5′ to 3′ direction, a sequence complementary to the first adapter, a sample tag, a UMI, and a linear amplification annealing site)) with a linear amplification primer 1061, the linear amplification primer having a sequence that is complementary to a linear amplification annealing site, to form annealed complexes 1065 and 1066. In some embodiments, a method may include contacting 1070 the annealed complexes 1065 and 1066 with a polymerase to form primer extension products 1071 complementary to the plus strand 1051 and primer extension products 1072 complementary to the minus strand 1052. Such annealing 1060 and primer extension 1070 may be repeated as desired (e.g., for a total of 2 to 200 cycles) to amplify each strand (1051, 1052) of the polynucleotide duplexes comprising double-stranded fragmented polynucleotides and 3′ overhangs, in which case, the primer extension products may collectively be referred to as amplification products. The amplification here would be linear since each strand of the polynucleotide duplexes has only one linear annealing site. The resulting primer extension products constitute a linearly amplified population of polynucleotides corresponding to the original fragmentation products. Primer extension products may comprise, in a 5′ to 3′ direction, a copy of the primer, a sample tag, a UMI, a sequence complimentary to a portion of the adapter, and a copy of the plus strand of the double-stranded fragmented polynucleotide or a copy of the minus strand of the double-stranded fragmented polynucleotide. A method 1000 may optionally comprise pooling 1075 primer extension products (e.g., primer extension products arising from workflows without a prior pooling step) after linear amplification.

In some embodiments, primer extension products may be interrogated for the presence of polynucleotides having a sequence of interest (a “target sequence”). For example, a method may comprise contacting 1080 primer extension products with target isolation probes (1081, 1082) to form a mixture comprising the primer extension products and target isolation probes. A mixture may further comprise, if target sequences are present, target complexes 1085, 1086 comprising primer extension products having the target sequence hybridized (e.g., specifically hybridized) to the target-specific oligonucleotides of the target isolation probes. Target isolation probes (baits) may comprise an affinity domain (e.g., biotin) and a polynucleotide, the polynucleotide having either a polynucleotide sequence complementary to the plus strand of the sequence of interest (e.g., a sequence of interest anywhere within the fragmented polynucleotide, for example, at or near the 5′ or 3′ end of the plus strand of the fragmented polynucleotide) or a polynucleotide sequence complementary to the minus strand of the sequence of interest (e.g., a sequence of interest anywhere within the fragmented polynucleotide, for example, at or near the 5′ or 3′ end of the minus strand of the fragmented polynucleotide). The conditions of contacting 1080 may be adjusted as desired, for example, to increase or decrease the stringency of bait and target sequence hybridization.

Optionally, a method 1000 may comprise contacting 1090 the mixtures potentially comprising target complexes with a 3′ single stranded exonuclease to remove any 3′ overhang and form an end (e.g., a blunt end) defined by the 3′ end of target sequence and the 5′ end of the bait.

In some embodiments, a method 1000 may include contacting 1100 mixtures potentially comprising target complexes 1085, 1086 with a solid support 1101 (e.g., magnetic beads), the solid support comprising affinity capture domains (e.g., streptavidin) corresponding to the affinity domains (e.g., biotin) of the target isolation probes. Contacting 1100 may comprise binding affinity domains (e.g., biotin) to their corresponding affinity capture domains (e.g., streptavidin) to form affinity capture complexes 1105 comprising affinity domains bound to affinity capture domains. Contacting 1100 may be performed to enrich primer extension products having a target sequence. For example, if the affinity domain is biotin, target sequences may be enriched by binding affinity domain (biotin) to a support comprising streptavidin affinity capture domains (e.g., beads). In some embodiments, magnetic beads coated in streptavidin may be added to the reaction mix after hybridization of primer extension products to target-specific oligonucleotides.

In some embodiments, a method 1000 may optionally comprise enriching 1110 affinity capture complexes 1105, for example, by securing solid support, for example, with magnets for magnetic beads, and/or washing to remove unbound affinity capture domains. The magnetic beads may be isolated by magnetism and then washed, thereby enriching for complexes that comprise primer extension products having the target sequence.

A method 1000 may comprise contacting 1120 blunt-ended target complexes with a second adapter, for example, a blunt ended adapter 1121. A blunted ended adapter 1121 may have a top strand 1121a and a bottom strand 1121b. A top strand 1121a may comprise, in a 5′-3′ direction, a 5′ phosphate, a sequence that is complementary to the adapter bottom strand and a nuclease resistant 3′end. A blunt ended adapter nuclease resistant 3′ end may comprise one or more phosphorothiate internucleotide linkages and/or a 3′ C3 spacer (Integrated DNA Technologies, Inc.). A top strand 1121a may optionally comprise a sample tag (optionally, the same or different from the sample tag of adapter 1033). A blunt ended adapter bottom strand 1121b may be shorter than the top strand and include deoxyuridines with no 5′ phosphate, no sample tag, no UMI, and no linear amplification annealing site. A blunt ended adapter bottom strand 1121b may include a sequence complementary to at least a portion of the 5′ end of the top strand 1121a. Contacting 1120 may further comprise contacting blunt-ended target complexes and blunt ended adapter 1121 with a ligase to form target complex-adapter ligation products. Ligation products may be washed, for example, to remove unligated adapter from target complex-adapter ligation products.

A method 1000 may comprise contacting 1130 target complex-adapter ligation products with an amplification primer comprising, in a 5′ to 3′ direction, a sequence complementary to the blunt ended adapter and a sample tag (optionally, the same or different from the sample tag of adapter 1033) shown as an index 1 (i7) sequence. Contacting 1130 may further comprise amplifying (e.g., by PCR, LAMP) to form a population of polynucleotides enriched (e.g., relative to the original population of fragmented polynucleotides) with target sequences, which population may be referred to as a final amplified library. A final amplified library may be sequenced, for example, using available next generation sequencing protocols (e.g., Illumina).

### Example 2

In some embodiments, enrichment methods may be practiced in accordance with the example shown in FIG. 2A and FIG. 2B. As illustrated, a method 2000 may comprise attracting 2030 (e.g., by bead-linked tagmentation or in-solution tagmentation) polynucleotides (e.g., unfragmented polynucleotides) a first adapter (e.g., double-stranded adapter, a single-stranded hairpin adapter). In some embodiments, a first adapter, adapter 2033, may comprise a top strand 2034 and a bottom strand 2035. A double-stranded adapter, in some embodiments, may comprise a plurality of deoxyuridines in the top strand, in the bottom strand or in both the top and bottom strands. A top strand 2034 of adapter 2033 may comprising, in a 5′ to 3′ direction, a sequence complementary to a linear amplification annealing site (“LAAS” in FIG. 2B), optionally a sample tag (e.g., i5), a unique molecular identifier (UMI), and a sequence complementary to the bottom strand 2034 (e.g., comprising a transposase binding sequence). A bottom strand 2035 of adapter 2033 may comprise, in a 5′ to 3′ direction, a 5′-OH (e.g., a 5′ terminus lacking a phosphate), a sequence complementary to the top strand and a non-extendible modified nucleotide (e.g., an inverted dT). Contacting 2030, in some embodiments, may comprise ligating the 3′ end of the adapter top strand to the 5′ end of the fragmented polynucleotides without ligating the 5′ end of the adapter bottom strand to the 3′ end of the fragmented polynucleotides to form double-stranded ligation products comprising a plus strand 2031 comprising a single-stranded break or nick and a minus strand 2032 comprising a single-stranded break or nick.

According to some embodiments, a method 2000 may comprise contacting 2040 double-stranded ligation products comprising nicks 2046 and 2047, with a nick-translating or strand displacing polymerase to form a nick-translated duplex polynucleotide comprising a plus strand 2041 and a minus strand 2042. Examples of nick translating polymerases include E. coli DNA polymerase I, pol Ik, Taq DNA polymerase, OneTaq® DNA polymerase (New England Biolabs, Inc.), Bst DNA polymerase (e.g., large fragment), Bsu DNA polymerase, phi29 DNA polymerase, Therminator™ DNA Polymerase, Klenow, Vent® DNA polymerase, and Deep Vent® DNA polymerase.

A method 2000 may include, in some embodiments, contacting 2050 the nick-translated duplex polynucleotide (e.g., comprising one or more modified nucleotides) with a single nucleotide excision reagent to form polynucleotide duplexes comprising a plus strand 2051 and a minus strand 2052, each comprising gaps (e.g., previously occupied by modified nucleotides) defining oligonucleotides of varying (e.g., generally short) lengths. For example, a plus strand 2051 of formed polynucleotide duplexes may comprise, in a 5′ to 3′ direction, a portion having gaps (e.g., single nucleotide previously occupied by U), a plus strand of the double-stranded fragmented polynucleotide, and a portion comprising a sequence complementary to the first adapter, a UMI, a sample tag, and a linear amplification annealing site.

Examples of a single nucleotide excision reagent may comprise an enzyme preparation that achieves uracil removal and phosphodiester bond cleavage (e.g., a USER® enzyme M5505 or M5508 (New England Biolabs, Inc.)) or may comprise a combination of enzymes (e.g., uracil DNA glycosylase in combination with endonuclease VIII. It will be appreciated that some polynucleotide sequences may comprise deoxyuridines at consecutive sequences. Treatment with a single nucleotide excision reagent may result in corresponding consecutive single nucleotide gaps, that is gaps of 2 or more nucleotides, according to the number of consecutive deoxyuridines originally present. A method may optionally include removal of the oligonucleotides formed by the treatment with the single nucleotide excision reagent.

A method 2000 may comprise contacting 2060 each strand (1051, 2052) of tagged fragments (polynucleotide duplexes comprising double-stranded fragmented polynucleotides and 3′ overhangs (e.g., 3′ overhangs comprising, in a 5′ to 3′ direction, a sequence complementary to the first adapter, a sample tag, a UMI, and a linear amplification annealing site)) with a linear amplification primer 2061, the linear amplification primer having a sequence that is complementary to a linear amplification annealing site, to form annealed complexes 2065 and 2066. In some embodiments, a method may include contacting 2070 the annealed complexes 2065 and 2066 with a polymerase to form primer extension products 2071 complementary to the plus strand 2051 and primer extension products 2072 complementary to the minus strand 2052. Such annealing 2060 and primer extension 2070 may be repeated as desired (e.g., for a total of 2 to 200 cycles) to amplify each strand (1051, 2052) of the polynucleotide duplexes comprising double-stranded fragmented polynucleotides and 3′ overhangs, in which case, the primer extension products may collectively be referred to as amplification products. The amplification here would be linear since each strand of the polynucleotide duplexes has only one linear annealing site. The resulting primer extension products constitute a linearly amplified population of polynucleotides corresponding to the original fragmentation products. Primer extension products may comprise, in a 5′ to 3′ direction, a copy of the primer, a sample tag, a UMI, a sequence complimentary to a portion of the adapter, and a copy of the plus strand of the double-stranded fragmented polynucleotide or a copy of the minus strand of the double-stranded fragmented polynucleotide. A method 2000 may optionally comprise pooling 2075 primer extension products (e.g., primer extension products arising from workflows without a prior pooling step) after linear amplification.

In some embodiments, primer extension products may be interrogated for the presence of polynucleotides having a sequence of interest (a “target sequence”). For example, a method may comprise contacting 2080 primer extension products with target isolation probes (1081, 2082) to form a mixture comprising the primer extension products and target isolation probes. A mixture may further comprise, if target sequences are present, target complexes 2085, 2086 comprising primer extension products having the target sequence hybridized (e.g., specifically hybridized) to the target-specific oligonucleotides of the target isolation probes. Target isolation probes (baits) may comprise an affinity domain (e.g., biotin) and a polynucleotide, the polynucleotide having either a polynucleotide sequence complementary to the plus strand of the sequence of interest (e.g., a sequence of interest anywhere within the fragmented polynucleotide, for example, at or near the 5′ or 3′ end of the plus strand of the fragmented polynucleotide) or a polynucleotide sequence complementary to the minus strand of the sequence of interest (e.g., a sequence of interest anywhere within the fragmented polynucleotide, for example, at or near the 5′ or 3′ end of the minus strand of the fragmented polynucleotide). The conditions of contacting 2080 may be adjusted as desired, for example, to increase or decrease the stringency of bait and target sequence hybridization.

Optionally, a method 2000 may comprise contacting 2090 the mixtures potentially comprising target complexes with a 3′ single stranded exonuclease to remove any 3′ overhang and form an end (e.g., a blunt end) defined by the 3′ end of target sequence and the 5′ end of the bait.

In some embodiments, a method 2000 may include contacting 2100 mixtures potentially comprising target complexes 2085, 2086 with a solid support 2101 (e.g., magnetic beads), the solid support comprising affinity capture domains (e.g., streptavidin) corresponding to the affinity domains (e.g., biotin) of the target isolation probes. Contacting 2100 may comprise binding affinity domains (e.g., biotin) to their corresponding affinity capture domains (e.g., streptavidin) to form affinity capture complexes 2105 comprising affinity domains bound to affinity capture domains. Contacting 2100 may be performed to enrich primer extension products having a target sequence. For example, if the affinity domain is biotin, target sequences may be enriched by binding affinity domain (biotin) to a support comprising streptavidin affinity capture domains (e.g., beads). In some embodiments, magnetic beads coated in streptavidin may be added to the reaction mix after hybridization of primer extension products to target-specific oligonucleotides.

In some embodiments, a method 2000 may optionally comprise enriching 2110 affinity capture complexes 2105, for example, by securing solid support, for example, with magnets for magnetic beads, and/or washing to remove unbound affinity capture domains. The magnetic beads may be isolated by magnetism and then washed, thereby enriching for complexes that comprise primer extension products having the target sequence.

A method 2000 may comprise contacting 2120 blunt-ended target complexes with a second adapter, for example, a blunt ended adapter 2121. A blunted ended adapter 2121 may have a top strand 2121a and a bottom strand 2121b. A top strand 2121a may comprise, in a 5′-3′ direction, a 5′ phosphate, a sequence that is complementary to the adapter bottom strand and a nuclease resistant 3′end. A blunt ended adapter nuclease resistant 3′ end may comprise one or more phosphorothiate internucleotide linkages and/or a 3′ C3 spacer (Integrated DNA Technologies, Inc.). A top strand 2121a may optionally comprise a sample tag (optionally, the same or different from the sample tag of adapter 2033). A blunt ended adapter bottom strand 2121b may be shorter than the top strand and include deoxyuridines with no 5′ phosphate, no sample tag, no UMI, and no linear amplification annealing site. A blunt ended adapter bottom strand 2121b may include a sequence complementary to at least a portion of the 5′ end of the top strand 2121a. Contacting 2120 may further comprise contacting blunt-ended target complexes and blunt ended adapter 2121 with a ligase to form target complex-adapter ligation products. Ligation products may be washed, for example, to remove unligated adapter from target complex-adapter ligation products.

A method 2000 may comprise contacting 2130 target complex-adapter ligation products with an amplification primer comprising, in a 5′ to 3′ direction, a sequence complementary to the blunt ended adapter and a sample tag (optionally, the same or different from the sample tag of adapter 2033) shown as an index 1 (i7) sequence. Contacting 2130 may further comprise amplifying (e.g., by PCR, LAMP) to form a population of polynucleotides enriched (e.g., relative to the original population of fragmented polynucleotides) with target sequences, which population may be referred to as a final amplified library. A final amplified library may be sequenced, for example, using available next generation sequencing protocols (e.g., Illumina).

### Example 3: An Enriched Library of Target Sequences from Human DNA Using Linear Amplification

Human gDNA was added to NEBNext Ultra II End Repair/dA-Tailing enzyme mix and buffer according to manufacturer's instructions (NEB). The mixture was cooled to 4° C., and a double-stranded adapter template (the first adapter) was added, with the top strand being 5′ A*A*T* GAU ACG GCG ACC ACC GAG AUC UAC ACT ATA GCC TNN NNN NNN NNN NAC ACU CTT TCC CUA CAC GAC GCU CTT CCG AUC* T (SEQ ID NO: 1), wherein the asterisks represent phosphorothioate linkages and the bold, underlined text represents an example sample tag.

A 12N (a random sequence of 12 nucleotides) UMI, a sample tag (the 8 bold underlined letters) and several deoxyuridine residues were present in the adapter. NEBNext Ultra II ligation master mix was added to the reaction mixture and incubated for 15 minutes at 20° C., followed by 15 minutes at 65° C. A nick-translating polymerase and dNTPS were added and incubated for an additional 45 minutes at 65° C., followed by 20 minutes at 80° C. After cooling to room temperature, USER enzyme was added, followed by an incubation of 15 minutes at 37° C. The DNA fragments were purified using NEB sample purification beads and eluted in nuclease free water.

DNA fragments in water were added to NEBNext Hot Start HiFi 2× Master Mix and linear amplification primer, with sequence 5′ AATGATACGGCGACCACC 3′ (SEQ ID NO: 2), wherein the 3′OH is replaced by a 3′ C3 spacer (Integrated DNA Technologies, Inc.). The reaction was incubated at 98° C. for 30 seconds, and then subjected to 20 cycles of 98° C. for 10 seconds, 55° C. for 45 seconds, and 72° C. for 20 seconds, then a final incubation at 72° C. for 5 minutes (step 3 of FIGS. 1A-1B).

This reaction was transferred to a hybridization mix that contained target isolation probes, each comprising a target-specific oligonucleotide (bait) and an affinity domain (namely, biotin), and incubated at 95° C. for 10 minutes, then 58° C. for 16 hours (step 4 of FIGS. 1A-1B). A target-specific oligonucleotide was designed to bind to the 3′ end of the target region. After hybridization, the target isolation probe/target DNA complexes were bound to NEB hydrophilic streptavidin magnetic beads for 10 minutes at 48° C., then washed twice for 5 minutes at 62° C. with a wash buffer (step 5 of FIGS. 1A-1B).

The beads were resuspended in a 3′-5′ single stranded exonuclease buffer with enzyme and incubated for 10 minutes at 25° C., and 5 minutes at 37° C. (step 5 of FIGS. 1A-1B). The beads were then washed and resuspended in 1×NEB Quick ligation buffer. NEB Quick Ligase, and a 3′ adapter for ligation to the 3′ end of the target sequence were added to the buffer and incubated for 15 minutes at 20° C. (step 6 of FIGS. 1A-1B). This 3′ adapter had a protective modification on the 3′ end of the top strand and a bottom complementary strand with modified bases. The 3′ adapter had a top strand sequence 5′ 5Phos/AGATC GGAAG AGCAC ACGTC TGAAC TCC*A*G*T*C*A*C/3SpC3 3′ (SEQ ID NO: 3) and a bottom strand sequence 5′ ACGNG TGCNC TNCCG ANC*T 3′ in which N is ideoxyU (SEQ ID NO:4). The beads were then washed and resuspended in 100 μl of 1×NEBuffer 1 containing Endonuclease VIII and UDG and incubated for 15 minutes at 37° C. (1090 of FIGS. 1A-1B).

The magnetic beads were then washed and resuspended in a 1×NEBNext Q5 Hot Start HiFi PCR Master Mix containing NEBNext Direct® PCR primers (New England Biolabs, Ipswich, MA) for PCR amplification.

### Example 4: Analysis of Enriched Libraries

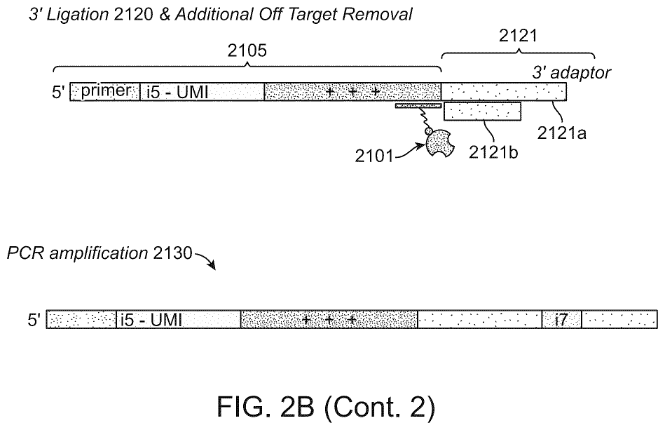

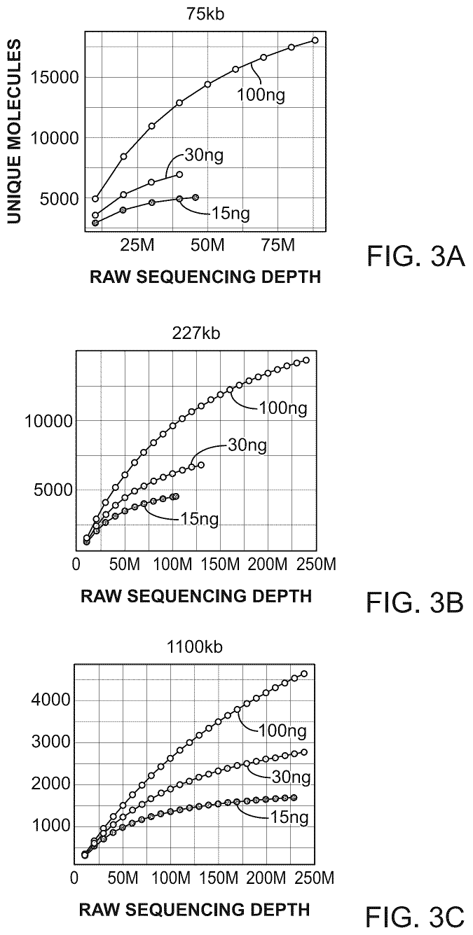

Enriched libraries were prepared in accordance with Example 1 beginning with fragmented human DNA inputs of 15, 30, and 100 ng and sequenced with paired end reads of 75 bp. The raw sequencing files were demultiplexed using the Picard tool ExtractllluminaBarcodes to assign reads to sample indexes followed by Picard's IlluminaBasecallsToSam which creates an unmapped BAM file containing the reads for each sample (“Picard Toolkit” 2019, Broad Institute, GitHub Repository). Unaligned BAMs were aligned to the human reference genome (build GRCh38/hg38) using bwa mem(Li H., 2013, Aligning sequence reads, clone sequences and assembly contigs with BWA-MEM. arXiv:1303.3997v2 [q-bio.GN]). Sequencing duplicates were then identified with Picard's MarkDuplicates using both position and unique molecular indexes (UMIs) to make the identification. The duplicate marked BAM is then used in variant calling. Germline calls are made using the GATK's HaplotypeCaller while somatic calls are made using both the GATK's MuTect2 and AstraZeneca's VarDict (Poplin R, et al., 2017, Scaling accurate genetic variant discovery to tens of thousands of samples bioRxiv, 201178. DOI: 10.1101/201178; “GATK Mutect2” 2019, Broad Institute, GitHub Repository; David Benjamin et al., 2019, Calling Somatic SNVs and Indels with Mutect2. bioRxiv 861054; doi: https://doi.org/10.1101/861054; Zhongwu Lai et al., VarDict: a novel and versatile variant caller for next-generation sequencing in cancer research, Nucleic Acids Research, Volume 44, Issue 11, 20 Jun. 2016, Page e108, https://doi.org/10.1093/nar/gkw227). QC metrics are calculated from the duplicate marked BAM using a combination of Picard and custom tools. Sequencing depths of coverage across numbers of reads for these target enrichment libraries are shown in FIG. 3A (75 kb target), FIG. 3B (227 kb target), and FIG. 3C (1100 kb target).

What is claimed is:

1. A method, comprising:
(a) fusing a DNA adapter to 5′ ends of double-stranded polynucleotide fragments of a population of fragments, wherein the DNA adapter comprises a bottom strand and a top strand, wherein:
(i) the adapter top strand comprises, from 5′ to 3′:
a sequence of at least 8 nucleotides that is complementary to a linear amplification annealing site,
optionally, a sample tag,
optionally, a unique molecule identifier (UMI); and
a sequence that is complementary to the adapter bottom strand; and
(ii) the adapter bottom strand comprises a non-extendible 3′ end,
wherein one or more of the polynucleotide fragments comprise a target sequence and wherein fusion products are formed, the fusion products comprising 3′ ends of the adapter top strands fused to 5′ ends of the polynucleotide fragments and nicks comprising unfused 5′ ends of the adapter bottom strands and 3′ ends of the polynucleotide fragments;
(b) contacting the fusion products with a nick-translating polymerase for templated addition of nucleotides to the 3′ ends of the polynucleotide fragments to form nick translation products comprising 3′ extensions of the polynucleotide fragments, the extensions complementary to the adapter top strands; and
(c) contacting the nick translation products with a primer having a sequence complementary to the linear amplification annealing site and a polymerase to produce linearly amplified nick translation products, the linearly amplified nick translation products comprising, in a 5′ to 3′ direction:
a sequence complimentary to the linear amplification annealing site,
optionally, a sequence of the sample tag,
optionally, a sequence of the UMI,
a sequence of a portion of the adapter top strand,
a sequence of one of the polynucleotide fragments, and
optionally, a sequence complementary to a portion of the adapter bottom strand.

2. The method of claim 1, further comprising, prior to or concurrent with (c), contacting nick translation products with a glycosylase and an endonuclease, wherein the adapter top strand template further comprises one or more modified nucleotides, wherein the one or more modified nucleotides are optionally deoxyuridine, the glycosylase is optionally uracil-DNA glycosylase (UDG), and the endonuclease is optionally endonuclease VIII.

3. The method of claim 1, further comprising removing the adapter bottom strand of the adapter template after (1b) by cleaving the adapter bottom strand with a reagent comprising a glycosylase and an endonuclease, wherein the adapter bottom strand comprises one or more modified nucleotides, wherein the one or more modified nucleotides are optionally deoxyuridine, the glycosylase is optionally uracil-DNA glycosylase (UDG), and the endonuclease is optionally endonuclease VIII.

4. The method of claim 1, wherein the adapter bottom strand non-extendible 3′ end comprises an inverted deoxythymidine.

5. The method of claim 1, further comprising:
(d) contacting the linearly amplified nick translation products with a target-specific oligonucleotide attached to an affinity domain to form target complexes comprising polynucleotide fragments having the target sequence, the target-specific oligonucleotide, and the affinity domain, wherein the target sequence is hybridized to the target-specific oligonucleotide.

6. The method of claim 5, further comprising:
(e) binding the target complexes to a solid support comprising an affinity capture domain corresponding to the affinity domain.

7. The method of claim 6, wherein the affinity domain is biotin and the affinity capture domain comprises streptavidin.

8. The method according to claim 5, further comprising,
(e) optionally binding the target complexes to a solid support comprising an affinity capture domain corresponding to the affinity domain;
(f) removing any overhanging polynucleotide sequence at the 3′ end of the oligonucleotide hybridized complementary strand using a 3′-5′ single strand exonuclease or a plurality of 3′-5′ exonucleases for forming a flush end duplex of the 3′ end of the complement and the 5′ end of the oligonucleotide;
(g) fusing the flush end duplex to a second adapter comprising a second sample tag to form second fusion products comprising, in a 5′ to 3′ direction, the first adapter top strand, the oligonucleotide fragment, and the second adapter; and
(h) amplifying the second fusion products for sequencing of one or both of the strands.

9. The method of claim 1, further comprising:
pooling the nick translation products with further nick translation products, the further nick translation products each comprising, in a 5′ to 3′ direction:
a sequence complimentary to the linear amplification annealing site,
the sequence of a further sample tag,
optionally, a sequence of the UMI,
a sequence of a portion of a further adapter top strand, and
a sequence of a polynucleotide fragment of a further population of fragments.

10. The method of claim 1, further comprising:
repeating step (a) with a second DNA adapter and double-stranded polynucleotide fragments of a second fragment population, wherein the first adapter top strand comprises a sample tag, and wherein the second adapter comprises a bottom strand and a top strand, wherein
(i) the second adapter top strand comprises, from 5′ to 3′:
a sequence of at least 8 nucleotides that is complementary to the linear amplification annealing site,
a sample tag that differs from the first adapter sample tag,
optionally, a unique molecule identifier (UMI); and
a sequence that is complementary to the second adapter bottom strand; and
(ii) the second adapter bottom strand comprises a non-extendible 3′ end,
wherein one or more of the polynucleotide fragments of the second population comprise a target sequence and wherein further fusion products are formed, the further fusion products comprising 3′ ends of the second adapter top strands fused to 5′ ends of the polynucleotide fragments of the second population and nicks comprising unfused 5′ ends of the second adapter bottom strands and 3′ ends of the polynucleotide fragments of the second population; and
pooling the fusion products and the further fusion products to form pooled fusion products.

11. The method of claim 10,
wherein the contacting the fusion products with the nick-translating polymerase further comprises contacting the pooled fusion products with the nick-translating polymerase for templated addition of nucleotides to the 3′ ends of the polynucleotide fragments to form pooled nick translation products comprising 3′ extensions of the polynucleotide fragments, the extensions complementary to the respective adapter top strands; and
wherein the contacting the nick translation products with the primer having a sequence complementary to the linear amplification annealing site further comprises contacting the pooled nick translation products with the primer and the polymerase to produce linearly amplified pooled nick translation products, the linearly amplified pooled nick translation products comprising, in a 5′ to 3′ direction:
a sequence complimentary to the linear amplification annealing site,
a sequence of one of the respective sample tags,
optionally, a sequence of the UMI,
a sequence of a portion of the respective adapter top strands,
a sequence of one of the polynucleotide fragments, and
optionally, a sequence complementary to a portion of the respective adapter bottom strands.

12. A method according to claim 1, wherein the fusing of step (a) comprises ligating or tagmentating the DNA adapter to the 5′ ends of the double-stranded polynucleotide fragments.

13. A method according to claim 1, wherein the top strand is longer than the adapter bottom strand.

14. A method according to claim 13, wherein the adapter top strand comprises at least one of the optional sample tag and the optional UMI.

15. A method according to claim 13, wherein the adapter bottom strand contains no 5′ phosphate, no sample tag, no UMI, and no linear amplification annealing site.

16. A method for enriching a target sequence, the method comprising:
(a) contacting a population of fragmented polynucleotides with a ligase and a first adapter, the first adapter comprising a bottom strand and a longer top strand, wherein
(i) the bottom strand comprises, in a 5′ to 3′ direction, a 5′-OH, at least 10 nucleotides complementary to the top strand, and optionally, an inverted dT; and
(ii) the top strand comprises, in a 5′ to 3′ direction, a sequence complementary to a linear amplification annealing site, optionally a sample tag, a unique molecular identifier (UMI), and a sequence complementary to the bottom strand,
wherein the top and bottom strands optionally and independently comprise one or more deoxyuridines and wherein (a) contacting produces double stranded fusion products comprising 3′ ends of adapter top strands fused to 5′ends of polynucleotide fragments and nicks comprising unfused 5′ ends of adapter bottom strands and 3′ ends of polynucleotide fragments;
(b) contacting the double stranded fusion products with a nick-translating polymerase and nucleotide triphosphates to form nick-translated duplex polynucleotides wherein the duplex comprises a top strand comprising, in a 5′ to 3′ direction, the adapter top strand, the plus strand of one of the polynucleotide fragments, and a sequence complementary to the adapter top strand and a bottom strand comprising, in a 5′ to 3′ direction, the adapter top strand, the minus strand of one of the polynucleotide fragments, and a sequence complementary to the adapter top strand;
(c) optionally contacting the nick-translated duplex polynucleotides with a single nucleotide excision reagent to form polynucleotide duplexes comprising a plus strand and a minus strand, each comprising single nucleotide gaps in the adapter top strand of the nick-translated duplex polynucleotides, the gaps defining oligonucleotides of varying lengths;
(d) contacting the nick-translated duplex polynucleotides with a linear amplification primer having a sequence that is complementary to the linear amplification annealing site to form annealed duplexes;
(e) contacting the annealed duplexes with a polymerase to form primer extension products comprising, in a 5′ to 3′ direction,
a sequence corresponding to at least a portion of the adapter top strand, and
a sequence corresponding to at least a portion of the plus strand of one of the polynucleotide fragments;
(f) repeating (d) contacting and (e) contacting at least once;
(g) contacting the primer extension products with a target isolation probe comprising an affinity domain and a target-specific oligonucleotide to form a target probe mixture comprising the primer extension products, target isolation probes, and if one or more target sequences are present in the population of fragmented polynucleotides, target complexes comprising primer extension products having the target sequence hybridized to the target-specific oligonucleotides of the target isolation probes;
(h) contacting the target probe mixture with a solid support comprising affinity capture domains corresponding to the affinity domains of the target isolation probes to form affinity complexes comprising affinity domains bound to affinity capture domains; and
(i) separating affinity complexes from unbound primer extension products and unbound target isolation probes to form an enriched target sequence composition.

17. A method according to claim 16, wherein the adapter top strand and the adapter bottom strand each comprise a plurality of deoxyuridines and the single nucleotide excision reagent removes deoxyuridines.

18. A method according to claim 16 further comprising, prior to (a) contacting, A-tailing the fragmented polynucleotides and wherein the top strand of the adapter further comprises a 3′ T or dU.

19. A method according to claim 16, wherein the first adapter includes the optional sample tag.

20. A method according to claim 19 further comprising repeating steps (a) through (i), wherein the population of fragmented polynucleotides of each repeated step (a) is different from all prior executions of step (a) and wherein the optional sample tag of each repeated step (a) is different from all prior executions of step (a).

21. A method according to claim 20 further comprising pooling the affinity complexes arising from the first execution of steps (a) through (i) with the affinity complexes arising from each repetition of steps (a) through (i).

22. A method according to claim 16, wherein the fusion of step (a) comprises ligation or tagmentation.

23. A method, comprising:
(a) fusing a DNA adapter to 5′ ends of double-stranded polynucleotide fragments of a population of fragments, wherein the DNA adapter comprises a top strand and optionally a bottom strand, wherein the adapter top strand comprises, from 5′ to 3′:
a sequence of at least 8 nucleotides that is complementary to a linear amplification annealing site,
optionally, a sample tag,
optionally, a unique molecule identifier (UMI); and
a sequence that is optionally complementary to the adapter bottom strand; and
wherein one or more of the polynucleotide fragments comprise a target sequence and wherein fusion products are formed, the fusion products comprising 3′ ends of the adapter top strands fused to 5′ ends of the polynucleotide fragments and nicks comprising 3′ ends of the polynucleotide fragments and optionally unfused 5′ ends of the adapter bottom strands;
(b) contacting the fusion products with a nick-translating polymerase for templated addition of nucleotides to the 3′ ends of the polynucleotide fragments to form nick translation products comprising 3′ extensions of the polynucleotide fragments, the extensions complementary to the adapter top strands; and
(c) contacting the nick translation products with a primer having a sequence complementary to the linear amplification annealing site and a polymerase to produce linearly amplified nick translation products, the linearly amplified nick translation products comprising, in a 5′ to 3′ direction:
a sequence complimentary to the linear amplification annealing site,
optionally, a sequence of the sample tag,
optionally, a sequence of the UMI,
a sequence of a portion of the adapter top strand,
a sequence of one of the polynucleotide fragments, and
optionally, a sequence complementary to a portion of the adapter bottom strand.

24. A method according to claim 23 wherein the adapter top strand lacks a UMI and the DNA adapter lacks a bottom strand.


SEQUENCE LISTING (4 sequences)

SEQ ID NO: 1 - DNA, 82 bp, synthetic construct
  misc_feature 1..82: note = Synthetic construct
  modified_base 1..2: mod_base = OTHER; note = phosphorothioate internucleotide linkage
  modified_base 2..3: mod_base = OTHER; note = phosphorothioate internucleotide linkage
  modified_base 3..4: mod_base = OTHER; note = phosphorothioate internucleotide linkage
  modified_base 6: mod_base = OTHER; note = n is uridine
  modified_base 23: mod_base = OTHER; note = n is uridine
  modified_base 25: mod_base = OTHER; note = n is uridine
  modified_base 38..48: mod_base = OTHER; note = n is adenine, cytosine, guanine, thymine or uridine
  misc_feature 49..56: note = example UMI
  modified_base 54: mod_base = OTHER; note = n is uridine
  modified_base 62: mod_base = OTHER; note = n is uridine
  modified_base 72: mod_base = OTHER; note = n is uridine
  modified_base 80: mod_base = OTHER; note = n is uridine
  modified_base 81..82: mod_base = OTHER; note = phosphorothioate internucleotide linkage
  source 1..82: mol_type = other DNA
aatganacggcgaccaccgagancnacactatagcctnnnnnnnnnnnnacacnctttcccnacacgacgcncttccganct

SEQ ID NO: 2 - DNA, 18 bp, synthetic construct
  misc_feature 1..18: note = Synthetic construct
  misc_feature 18: note = 3'OH is replaced by 3'OCH2CH2CH2OH
  source 1..18: mol_type = other DNA
aatgatacggcgaccacc

SEQ ID NO: 3 - DNA, 34 bp, synthetic construct
  misc_feature 1..34: note = Synthetic construct
  modified_base 28..29: mod_base = OTHER; note = phosphorothioate internucleotide linkage
  modified_base 29..30: mod_base = OTHER; note = phosphorothioate internucleotide linkage
  modified_base 30..31: mod_base = OTHER; note = phosphorothioate internucleotide linkage
  modified_base 31..32: mod_base = OTHER; note = phosphorothioate internucleotide linkage
  modified_base 32..33: mod_base = OTHER; note = phosphorothioate internucleotide linkage
  modified_base 33..34: mod_base = OTHER; note = phosphorothioate internucleotide linkage
  misc_feature 34: note = 3'OH is replaced by 3'OCH2CH2CH2OH
  source 1..34: mol_type = other DNA
agatcggaagagcacacgtctgaactccagtcac

SEQ ID NO: 4 - DNA, 19 bp, synthetic construct
  misc_feature 1..19: note = Synthetic construct
  modified_base 4: mod_base = OTHER; note = n is ideoxyU
  modified_base 9: mod_base = OTHER; note = n is ideoxyU
  modified_base 12: mod_base = OTHER; note = n is ideoxyU
  modified_base 17: mod_base = OTHER; note = n is ideoxyU
  modified_base 18..19: mod_base = OTHER; note = phosphorothioate internucleotide linkage
  source 1..19: mol_type = other DNA
acgngtgcnctnccganct